(19) United States
(12) Patent Application Publication
(10) Pub. No.: US 2024/0087147 A1
(43) Pub. Date: Mar. 14, 2024

(54) INTRAVASCULAR ULTRASOUND CO-REGISTRATION WITH ANGIOGRAPHIC IMAGES

(71) Applicant: Boston Scientific Scimed Inc., Maple Grove, MN (US)

(72) Inventors: Judith Tiferes Wang, Buenos Aires (AR); Brandon Zingsheim, Circle Pines, MN (US); Andrew Oveson, Bloomington, MN (US); Wenguang Li, Los Gatos, CA (US); Qian Li, San Jose, CA (US)

(21) Appl. No.: 18/466,677

(22) Filed: Sep. 13, 2023

Related U.S. Application Data
(60) Provisional application No. 63/375,562, filed on Sep. 14, 2022.

(51) Int. Cl. G06T 7/38; A61B 6/00; A61B 6/12; A61B 8/00; A61B 8/08; A61B 8/12; A61B 34/20

(52) U.S. Cl. CPC G06T 7/38; A61B 6/12; A61B 6/504; A61B 6/5247; A61B 8/12; A61B 8/467; A61B 8/5261; A61B 34/20; A61B 2034/2063; G06T 2207/10116; G06T 2207/10132; G06T 2207/30101

(57) ABSTRACT

The present disclosure provides to co-register an external image of a vessel to IVUS images captured within the vessel and provides graphical user interfaces to facilitate modification to the co-registration. Notably, the present disclosure provides for the co-registration even when the IVUS images are captured before the external image or before co-registration is initiated with the external image.

### CROSS-REFERENCE TO RELATED APPLICATIONS

This application claims the benefit of U.S. Provisional Patent Application Ser. No. 63/375,562 filed on Sep. 14, 2022, the disclosure of which is incorporated herein by reference.

### TECHNICAL FIELD

The present disclosure generally relates to intravascular ultrasound (IVUS) imaging system. Particularly, but not exclusively, the present disclosure relates to co-registering angiographic images with IVUS images and graphical user interfaces for the same.

### BACKGROUND

Ultrasound devices insertable into patients have proven diagnostic capabilities for a variety of diseases and disorders. For example, intravascular ultrasound (IVUS) imaging systems have been used as an imaging modality for diagnosing blocked blood vessels and providing information to aid medical practitioners in selecting and placing stents, selecting sites for an atherectomy procedure, or the like.

IVUS imaging systems includes a control module (with a pulse generator, an image acquisition and processing components, and a monitor), a catheter, and a transducer disposed in the catheter. The transducer-containing catheter is positioned in a lumen or cavity within, or in proximity to, a region to be imaged, such as a blood vessel wall or patient tissue in proximity to a blood vessel wall. The pulse generator in the control module generates electrical pulses that are delivered to the transducer and transformed to acoustic pulses that are transmitted through patient tissue. The patient tissue (or other structure) reflects the acoustic pulses and reflected pulses are absorbed by the transducer and transformed to electric pulses. The transformed electric pulses are delivered to the image acquisition and processing components and converted into images displayable on the monitor.

However, it can be difficult for physicians to correlate IVUS images with other external imaging of the vessel to be treated. For example, it is difficult to correlate the overall structure of the vessel depicted in an angiogram with the structure of the same vessel depicted in IVUS images. Thus, there is a need for systems and methods to co-register external images with IVUS images.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides a workflow, system, graphical user interface, and processes to co-register IVUS images with external images, such as, an angiogram image.

In some implementations, the present disclosure be embodied as a method, for example, a method for an intravascular ultrasound (IVUS) imaging system, comprising receiving an indication of an end location, the end location corresponding to a location of an intravascular ultrasound (IVUS) guide catheter distal tip on an external image, wherein the IVUS guide catheter is or was inserted into a vessel and the vessel is represented in the external image; registering, based in part on the location of the IVUS guide catheter distal tip and a start location, each of a plurality of frames of a series of IVUS images captured from within the vessel via an IVUS pullback operation to locations on the external image to generate a pullback path of the IVUS pullback operation and a branch, wherein the start location corresponding to a location of the start of the IVUS pullback operation; generating a graphical user interface (GUI) comprising indications of the external image, the start location, the end location, the pullback path, and the branch; receiving, via an input device and the GUI at least one modification to the pullback path and/or the branch; regenerating the GUI, the regenerated GUI comprising indications of the external image, the start location, the end location, the pullback path and/or the modified branch; and receiving, via the input device and the regenerated GUI a confirmation that the modified pullback path and/or the modified branch is confirmed.

Alternatively, or additionally in any of the embodiments of a method above the at least one modification is a modification to the pullback path and the method can comprise receiving, via the input device and the regenerated GUI a modification to the branch; and regenerating the GUI a second time, the second regenerated GUI comprising indications of the external image, the start location, the end location, the modified pullback path, and the modified branch.

Alternatively, or additionally in any of the embodiments of a method above the pullback path is registered to a longitudinal axis of the vessel.

Alternatively, or additionally in any of the embodiments of a method above the pullback path comprises a plurality of nodes and the at least one modification to the pullback path comprises a modification to a location of one or more of the plurality of nodes.

Alternatively, or additionally in any of the embodiments of a method above the modification to the branch changes a location of the branch along the longitudinal axis.

Alternatively, or additionally in any of the embodiments of a method above the branch is a first branch of a plurality of branches and the method can comprise changing the location of a second branch of the plurality of branches responsive to the modification.

Alternatively, or additionally in any of the embodiments of a method above the first regenerated GUI further comprises numerical or textual designations for the plurality of branches.

Alternatively, or additionally any of the embodiments of a method above can comprise retrieving an angiogram from a memory device, the angiogram comprising a plurality of frames; generating an external image selection GUI comprising an indication of a frame of the plurality of frames of the angiogram and at least one navigation button to scroll through plurality of frames; and receiving, via the input device and the external image selection GUI, an indication to select a one of the plurality of frames of the angiogram as the external image.

Alternatively, or additionally any of the embodiments of a method above can comprise receiving, from the input device, an indication of the start location.

Alternatively, or additionally any of the embodiments of a method above can comprise retrieving the series of IVUS images from the memory device.

Alternatively, or additionally any of the embodiments of a method above can comprise receiving, from the input device, an indication of the start location on an external image of the vessel; capturing the external image via external image acquisition circuitry; and capturing the series of IVUS images via IVUS image acquisition circuitry.

Alternatively, or additionally in any of the embodiments of a method above the external image is an x-ray image.

Alternatively, or additionally in any of the embodiments of a method above the regenerated GUI further comprises an indication of a longitudinal view of the series of IVUS images, the first regenerated GUI further comprises a slider to scroll along the longitudinal axis of the series of IVUS images and a slider marker disposed on the pullback path, and the slider marker is linked to the slider such that movement of the slider causes a corresponding movement of the slider marker on the pullback path.

In some implementations, the present disclosure be embodied as an apparatus, comprising a processor coupled to a memory, the memory comprising instructions executable by the processor, the processor configured to couple to an intravascular ultrasound (IVUS) imaging system and configured to execute the instructions, which instructions when executed cause the processor to implement the method of any combination of the examples above.

In some implementations, the present disclosure be embodied as at least one machine readable storage device, comprising a plurality of instructions that in response to being executed by a processor of an intravascular ultrasound (IVUS) imaging system cause the processor to implement the method of any combination of the examples above.

In some implementations, the present disclosure be embodied as an apparatus for an intravascular ultrasound (IVUS) imaging system, comprising a display; an interface configured to couple to an IVUS catheter; a processor coupled to the interface and the display; and a memory device comprising instructions, which when executed by the processor cause the IVUS imaging system to: receive an indication of an end location, the end location corresponding to a location of an intravascular ultrasound (IVUS) guide catheter distal tip on an external image, wherein the IVUS guide catheter is or was inserted into a vessel and the vessel is represented in the external image; register, based in part on the location of the IVUS guide catheter distal tip and a start location, each of a plurality of frames of a series of IVUS images captured from within the vessel via an IVUS pullback operation to locations on the external image to generate a pullback path of the IVUS pullback operation and a branch, wherein the start location corresponding to a location of the start of the IVUS pullback operation; generate a graphical user interface (GUI) comprising indications of the external image, the start location, the end location, the pullback path, and the branch; receive, via an input device and the GUI at least one modification to the pullback path and/or the branch; regenerate the GUI, the regenerated GUI comprising indications of the external image, the start location, the end location, the pullback path and/or the modified branch; and receive, via the input device and the regenerated GUI a confirmation that the modified pullback path and/or the modified branch is confirmed.

Alternatively, or additionally in any of the embodiments of an apparatus above, the at least one modification is a modification to the pullback path and the memory device can further comprise instructions that when executed by the processor cause the IVUS imaging system to receive, via the input device and the regenerated GUI a modification to the branch; and regenerate the GUI a second time, the second regenerated GUI comprising indications of the external image, the start location, the end location, the modified pullback path, and the modified branch.

Alternatively, or additionally in any of the embodiments of an apparatus above, the pullback path is registered to a longitudinal axis of the vessel.

Alternatively, or additionally in any of the embodiments of an apparatus above, the pullback path comprises a plurality of nodes and the at least one modification to the pullback path comprises a modification to a location of one or more of the plurality of nodes.

Alternatively, or additionally in any of the embodiments of an apparatus above, the modification to the branch changes a location of the branch along the longitudinal axis.

Alternatively, or additionally in any of the embodiments of an apparatus above, the branch is a first branch of a plurality of branches and the memory device can further comprise instructions that when executed by the processor cause the IVUS imaging system change the location of a second branch of the plurality of branches responsive to the modification.

Alternatively, or additionally in any of the embodiments of an apparatus above, the first regenerated GUI further comprises numerical or textual designations for the plurality of branches.

Alternatively, or additionally in any of the embodiments of an apparatus above, the memory device further comprises instructions that when executed by the processor cause the IVUS imaging system to: retrieve an angiogram from a memory device, the angiogram comprising a plurality of frames; generate an external image selection GUI comprising an indication of a frame of the plurality of frames of the angiogram and at least one navigation button to scroll through plurality of frames; and receive, via the input device and the external image selection GUI, an indication to select a one of the plurality of frames of the angiogram as the external image.

Alternatively, or additionally in any of the embodiments of an apparatus above, the memory device further comprises instructions that when executed by the processor cause the IVUS imaging system to receive, from the input device, an indication of the start location.

Alternatively, or additionally in any of the embodiments of an apparatus above, the memory device further comprises instructions that when executed by the processor cause the IVUS imaging system to: receive, from the input device, an indication of the start location on an external image of the vessel; capture the external image via external image acquisition circuitry; and capture the series of IVUS images via IVUS image acquisition circuitry.

In some implementations, the present disclosure be embodied as at least one machine readable storage device, comprising a plurality of instructions that in response to being executed by a processor of an intravascular ultrasound (IVUS) imaging system cause the processor to receive an indication of an end location, the end location corresponding to a location of an intravascular ultrasound (IVUS) guide catheter distal tip on an external image, wherein the IVUS guide catheter is or was inserted into a vessel and the vessel is represented in the external image; register, based in part on the location of the IVUS guide catheter distal tip and a start location, each of a plurality of frames of a series of IVUS images captured from within the vessel via an IVUS pullback operation to locations on the external image to generate a pullback path of the IVUS pullback operation and a branch, wherein the start location corresponding to a location of the start of the IVUS pullback operation; generate a graphical user interface (GUI) comprising indications of the external image, the start location, the end location, the pullback path, and the branch; receive, via an input device and the GUI at least one modification to the pullback path and/or the branch; regenerate the GUI, the regenerated GUI comprising indications of the external image, the start location, the end location, the pullback path and/or the modified branch; and receive, via the input device and the regenerated GUI a confirmation that the modified pullback path and/or the modified branch is confirmed.

Alternatively, or additionally in any of the embodiments of an at least one machine readable storage device above, the instructions in response to being executed by the processor can further cause the processor to: receive, via the input device and the regenerated GUI a modification to the branch; and regenerate the GUI a second time, the second regenerated GUI comprising indications of the external image, the start location, the end location, the modified pullback path, and the modified branch.

Alternatively, or additionally in any of the embodiments of an at least one machine readable storage device above, the instructions in response to being executed by the processor can further cause the processor to receive, from the input device, an indication of the start location on an external image of the vessel; capture the external image via external image acquisition circuitry; and capture the series of IVUS images via IVUS image acquisition circuitry.

Alternatively, or additionally in any of the embodiments of an at least one machine readable storage device above, the external image is an x-ray image.

Alternatively, or additionally in any of the embodiments of an at least one machine readable storage device above, the regenerated GUI further comprises an indication of a longitudinal view of the series of IVUS images, the first regenerated GUI further comprises a slider to scroll along the longitudinal axis of the series of IVUS images and a slider marker disposed on the pullback path, and the slider marker is linked to the slider such that movement of the slider causes a corresponding movement of the slider marker on the pullback path.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a IVUS imaging system.

FIG. 2 illustrates an angiographic image of a vessel.

FIG. 3A illustrates a longitudinal view of IVUS images.

FIG. 3B illustrates a cross-section view of a frame of IVUS images.

FIG. 4 illustrates a combined IVUS/external imaging system.

FIG. 5 illustrates IVUS co-registration visualization system.

FIG. 6 illustrates a logic flow for an IVUS co-registration visualization system.

FIG. 7A illustrates a first graphical interface for an IVUS co-registration visualization system.

FIG. 7B illustrates a second view of the graphical interface of FIG. 7A.

FIG. 7C illustrates a third view of the graphical interface of FIG. 7A.

FIG. 7D illustrates a fourth view of the graphical interface of FIG. 7A.

FIG. 7E illustrates a fifth view of the graphical interface of FIG. 7A.

FIG. 7F illustrates a sixth view of the graphical interface of FIG. 7A.

FIG. 7G illustrates a second graphical interface for an IVUS co-registration visualization system.

FIG. 8 illustrates a first method for co-registration

FIG. 9 illustrates a second method for co-registration.

FIG. 10 illustrates a third method for co-registration.

FIG. 11A illustrates a second graphical interface for an IVUS co-registration visualization system.

FIG. 11B illustrates a second view of the graphical interface of FIG. 11A.

FIG. 11C illustrates a third view of the graphical interface of FIG. 11A.

FIG. 11D illustrates a fourth view of the graphical interface of FIG. 11A.

FIG. 11E illustrates a fifth view of the graphical interface of FIG. 11A.

FIG. 11F illustrates a sixth view of the graphical interface of FIG. 11A.

FIG. 11G illustrates a seventh view of the graphical interface of FIG. 11A.

FIG. 11H illustrates an eighth view of the graphical interface of FIG. 11A.

FIG. 11I illustrates a ninth view of the graphical interface of FIG. 11A.

FIG. 11J illustrates a tenth view of the graphical interface of FIG. 11A.

FIG. 11K illustrates an eleventh view of the graphical interface of FIG. 11A.

FIG. 12A illustrates a third graphical interface for an IVUS co-registration visualization system.

FIG. 12B illustrates a second view of the graphical interface of FIG. 12A.

FIG. 12C illustrates a third view of the graphical interface of FIG. 12A.

FIG. 12D illustrates a fourth view of the graphical interface of FIG. 12A.

FIG. 12E illustrates a fifth view of the graphical interface of FIG. 12A.

FIG. 12F illustrates a sixth view of the graphical interface of FIG. 12A.

FIG. 12G illustrates a seventh view of the graphical interface of FIG. 12A.

FIG. 12H illustrates an eighth view of the graphical interface of FIG. 12A.

FIG. 13 illustrates a computer-readable storage medium.

FIG. 14 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to IVUS images captured within a lumen (e.g., vessels) of a patient as well as external images of the vessel, such as an angiogram and to co-registering the IVUS images with the angiogram image. Said differently, the present disclosure provides to map or correlate frames in the IVUS images with locations on the angiogram image. As such, an example IVUS imaging system, patient vessel, series of IVUS images, and combined IVUS/external imaging system are described.

Suitable IVUS imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,246,959; 7,306,561; and 6,945,938; as well as U.S. Patent Application Publication Numbers 2006/0100522; 2006/0106320; 2006/0173350; 2006/0253028; 2007/0016054; and 2007/0038111; all of which are incorporated herein by reference.

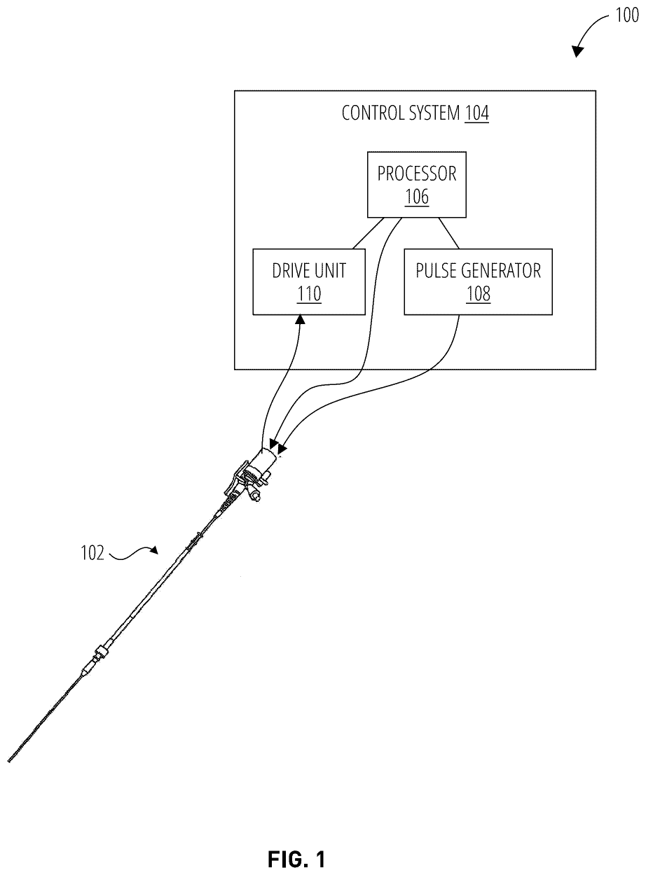

FIG. 1 illustrates schematically one embodiment of an IVUS imaging system 100. The IVUS imaging system 100 includes a catheter 102 that is couplable to a control system 104. The control system 104 may include, for example, a processor 106, a pulse generator 108, and a drive unit 110. In at least some embodiments, the pulse generator 108 forms electric pulses that may be input to one or more transducers (not shown) disposed in the catheter 102.

With some embodiments, mechanical energy from the drive unit 110 can be used to drive an imaging core (also not shown) disposed in the catheter 102. In at least some embodiments, electric signals transmitted from the one or more transducers may be input to the processor 106 for processing. In at least some embodiments, the processed electric signals from the one or more transducers can be used to form a series of images, described in more detail below. For example, a scan converter can be used to map scan line samples (e.g., radial scan line samples, or the like) to a two-dimensional Cartesian grid, which can be used as the basis for a series of IVUS images that can be displayed for a user.

In at least some embodiments, the processor 106 may also be used to control the functioning of one or more of the other components of the control system 104. For example, the processor 106 may be used to control at least one of the frequency or duration of the electrical pulses transmitted from the pulse generator 108, the rotation rate of the imaging core by the drive unit 110. Additionally, where IVUS imaging system 100 is configured for automatic pullback, the drive unit 110 can control the velocity and/or length of the pullback.

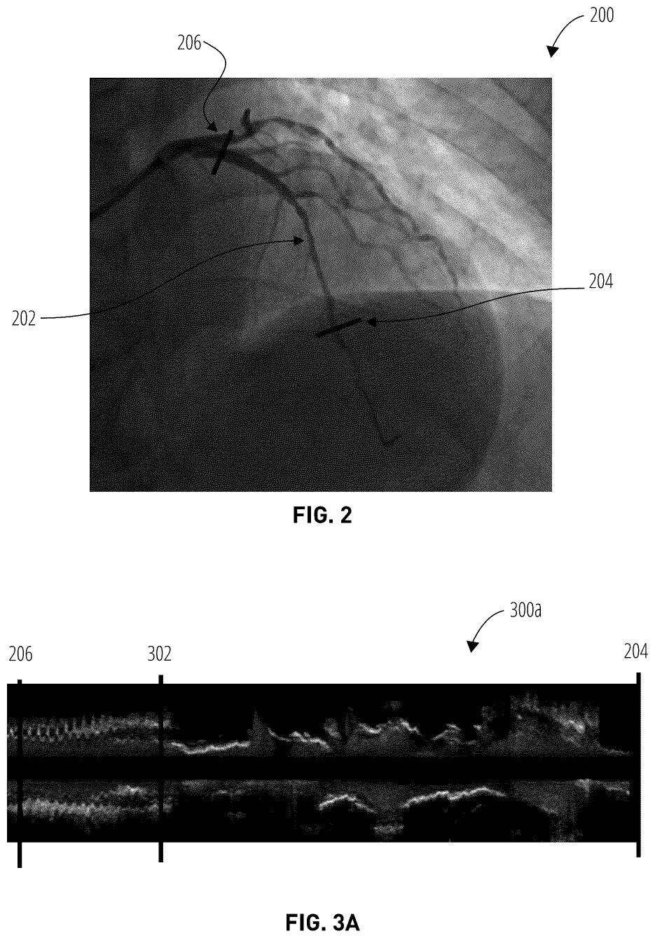

FIG. 2 illustrates an image 200 of a vessel 202 of a patient. As described, IVUS imaging systems (e.g., IVUS imaging system 100, or the like) are used to capture a series of images or a “recording” or a vessel, such as, vessel 202. For example, an IVUS catheter (e.g., catheter 102) is inserted into vessel 202 and a recording, or a series of IVUS images, is captured as the catheter 102 is pulled back from a distal end 204 to a proximal end 206. The catheter 102 can be pulled back manually or automatically (e.g., under control of drive unit 110, or the like).

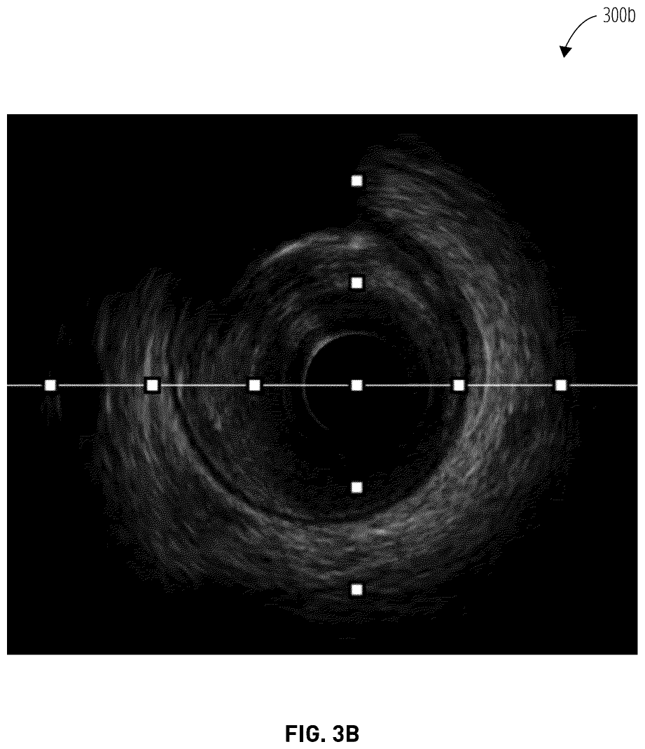

FIG. 3A and FIG. 3B illustrates two-dimensional (2D) representations of IVUS images of vessel 202. For example, FIG. 3A illustrates IVUS images 300a depicting a longitudinal view of the IVUS recording of vessel 202 between proximal end 206 and distal end 204.

FIG. 3B illustrates an image frame 300b depicting an on-axis (or short axis) view of vessel 202 at point 302. Said differently, image frame 300b is a single frame or single image from a series of IVUS images that can be captured between distal end 204 and proximal end 206 as described herein. As introduced above, the present disclosure provides systems and techniques to process raw IVUS images to identify regions of interest, such as, for example starting and ending points between which include frames of interest in a series of IVUS images.

For example, IVUS images 300a depicts an entire series of IVUS images taken of vessel 202 between distal end 204 and proximal end 206. However, not all these images may be of interest to a physician. The present disclosure provides to identify “key frames” such as, a proximal key frame and a distal key frame as well as a minimum key frame.

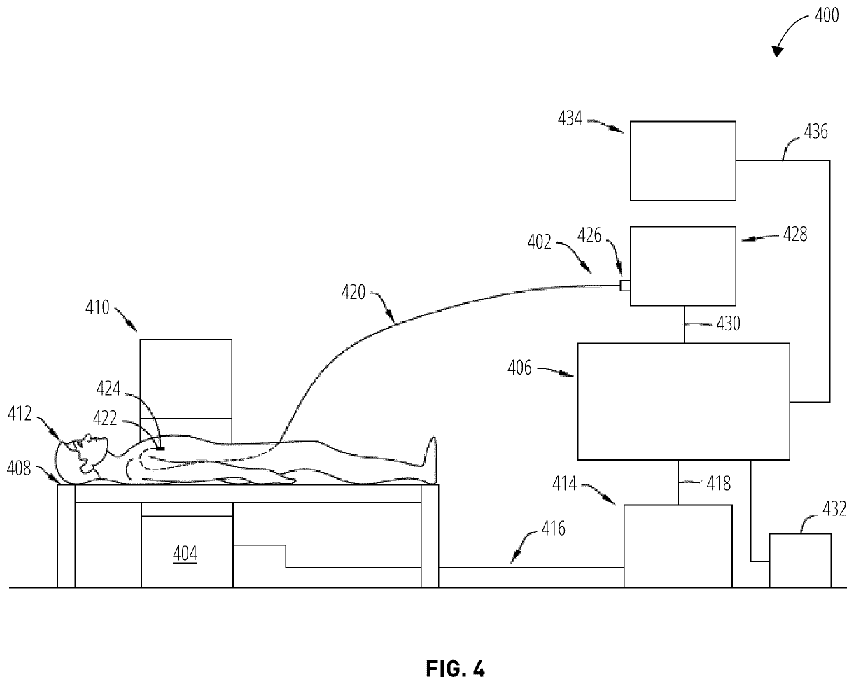

FIG. 4 illustrates a combined IVUS external imaging system 400 including both an IVUS imaging system 402 (e.g., IVUS imaging system 100, or the like) and an extravascular imaging system 404 (e.g., an angiographic imaging system). Combined IVUS external imaging system 400 further includes computing device 406, which includes circuitry, controllers, and/or processor(s) and memory and software configured to execute a method for vascular imaging registration of the obtained extravascular imaging data and the obtained intravascular imaging data. In general, the IVUS imaging system 402 can be arranged to generate IVUS intravascular imaging data (e.g., IVUS images 300a and image frame 300b) while the extravascular imaging system 404 can be arranged to generate extravascular imaging data (e.g., image 200).

The extravascular imaging system 404 may include an angiographic table 408 that may be arranged to provide sufficient space for the positioning of an angiography/fluoroscopy unit c-arm 410 in an operative position in relation to a patient 412 on the drive unit 110. Raw radiological image data acquired by the c-arm 410 may be passed to an extravascular data input port 414 via a transmission cable 416. The input port 414 may be a separate component or may be integrated into or be part of the computing device 406. The input port 414 may include a processor that converts the raw radiological image data received thereby into extravascular image data (e.g., angiographic/fluoroscopic image data), for example, in the form of live video, DICOM, or a series of individual images. The extravascular image data may be initially stored in memory within the input port 414 or may be stored within memory of computing device 406. If the input port 414 is a separate component from the computing device 406, the extravascular image data may be transferred to the computing device 406 through the transmission cable 418 and into an input port (not shown) of the computing device 406. In some alternatives, the communications between the devices or processors may be carried out via wireless communication, rather than by cables as depicted.

The intravascular imaging data may be, for example, IVUS data or OCT data obtained by the IVUS imaging system 402. The IVUS imaging system 402 may include an intravascular imaging device such as an imaging catheter 420. The imaging catheter 420 is configured to be inserted within the patient 412 so that its distal end, including a diagnostic assembly or probe 422 (e.g., an IVUS probe), is in the vicinity of a desired imaging location of a blood vessel. A radiopaque material or marker 424 located on or near the probe 422 may provide indicia of a current location of the probe 422 in a radiological image.

Imaging catheter 420 is coupled to a proximal connector 426 to couple imaging catheter 420 to image acquisition device 428. Image acquisition device 428 may be coupled to computing device 406 via transmission cable 430, or a wireless connection. The intravascular image data may be initially stored in memory within the image acquisition device 428 or may be stored within memory of computing device 406. If the image acquisition device 428 is a separate component from computing device 406, the intravascular image data may be transferred to the computing device 406, via, for example, transmission cable 430.

The computing device 406 can also include one or more additional output ports for transferring data to other devices. For example, the computer can include an output port to transfer data to a data archive or memory device 432. The computing device 406 can also include a user interface (described in greater detail below) that includes a combination of circuitry, processing components and instructions executable by the processing components and/or circuitry to enable dynamic co-registration of intravascular and extravascular images.

The user interface can be rendered and displayed on display 434 coupled to computing device 406 via display cable 436. Although the display 434 is depicted as separate from computing device 406, in some examples the display 434 can be part of computing device 406. Alternatively, the display 434 can be remote and wireless from computing device 406. As another example, the display 434 can be part of another computing device different from computing device 406, such as, a tablet computer, which can be coupled to computing device 406 via a wired or wireless connection.

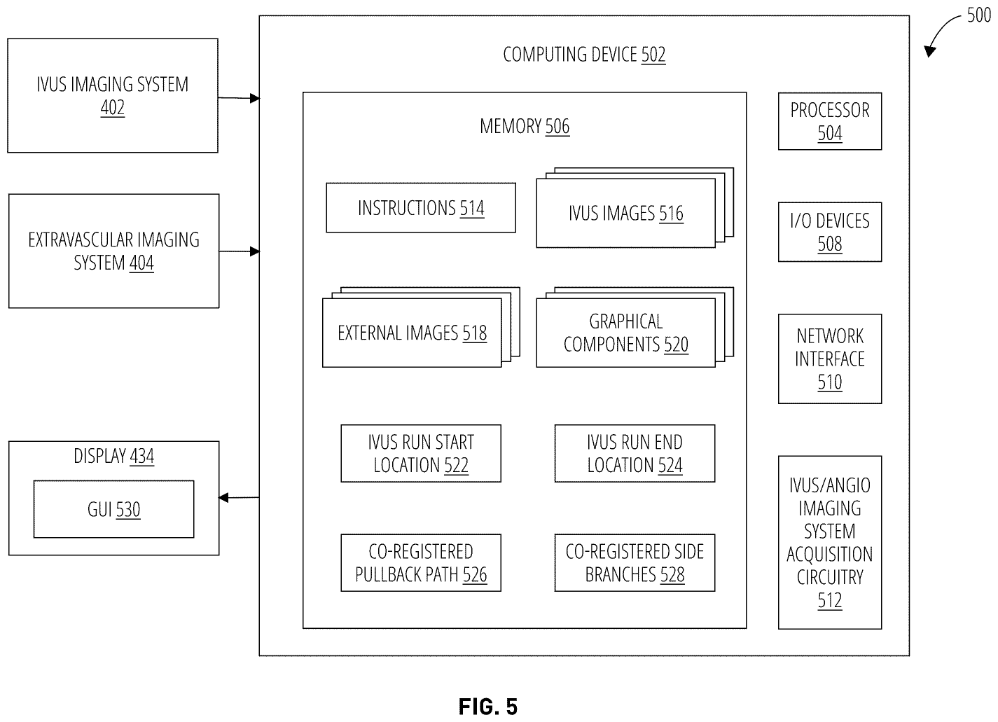

FIG. 5 illustrates a co-registration system 500, according to some embodiments of the present disclosure. In general, co-registration system 500 is a system for co-registering IVUS images with external images (e.g., an angiogram). Co-registration system 500 can be implemented in conjunction with a commercial IVUS guidance or navigation system, such as, for example, the AVVIGO® Guidance System available from Boston Scientific® and an external imaging system, such as, for example, the extravascular imaging system 404 described in conjunction with FIG. 4 above. The present disclosure provides advantages over prior or conventional IVUS imaging systems in that the external image can be co-registered whether the external image is captured along with the IVUS images or separately from the IVUS images. For example, the present disclosure can be implemented in an IVUS navigation system used in a percutaneous coronary intervention (PCI) where the external images are captured and co-registration is initiated in conjunction with capturing the IVUS images (e.g., at the time of pullback) or where the IVUS images and the external images are captured at different times. That is, co-registration can still be applied using the present disclosure. This is a significant advantage over current systems as a physician can still co-register images even after the images are captured.

Co-registration system 500 includes 502. Computing device 502 can be any of a variety of computing devices. In some embodiments, computing device 502 can be incorporated into and/or implemented by computing device 406. With some embodiments, computing device 502 can be a workstation or server communicatively coupled to IVUS imaging system 100 and an external imaging system (e.g., extravascular imaging system 404, or the like). With still other embodiments, computing device 502 can be provided by a cloud based computing device, such as, by a computing as a service system accessibly over a network (e.g., the Internet, an intranet, a wide area network, or the like). Computing device 502 can include processor 504, memory 506, input and/or output (I/O) devices 508, network interface 510, and IVUS/Angio imaging system acquisition circuitry 512.

The processor 504 may include circuitry or processor logic, such as, for example, any of a variety of commercial processors. In some examples, processor 504 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 504 may include graphics processing portions and may include dedicated memory, multiple-threaded processing and/or some other parallel processing capability. In some examples, the processor 504 may be an application specific integrated circuit (ASIC) or a field programmable integrated circuit (FPGA).

The memory 506 may include logic, a portion of which includes arrays of integrated circuits, forming non-volatile memory to persistently store data or a combination of non-volatile memory and volatile memory. It is to be appreciated, that the memory 506 may be based on any of a variety of technologies. In particular, the arrays of integrated circuits included in memory 120 may be arranged to form one or more types of memory, such as, for example, dynamic random access memory (DRAM), NAND memory, NOR memory, or the like.

I/O devices 508 can be any of a variety of devices to receive input and/or provide output. For example, I/O devices 508 can include, a keyboard, a mouse, a joystick, a foot pedal, a display, a touch enabled display, a haptic feedback device, an LED, or the like.

Network interface 510 can include logic and/or features to support a communication interface. For example, network interface 510 may include one or more interfaces that operate according to various communication protocols or standards to communicate over direct or network communication links. Direct communications may occur via use of communication protocols or standards described in one or more industry standards (including progenies and variants). For example, network interface 510 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 510 can include logic and/or features to enable communication over a variety of wired or wireless network standards (e.g., 802.11 communication standards). For example, network interface 510 may be arranged to support wired communication protocols or standards, such as, Ethernet, or the like. As another example, network interface 510 may be arranged to support wireless communication protocols or standards, such as, for example, Wi-Fi, Bluetooth, ZigBee, LTE, 5G, or the like.

The IVUS/Angio imaging system acquisition circuitry 512 may include circuitry including custom manufactured or specially programmed circuitry configured to receive or receive and send signals between IVUS imaging system 100 including indications of an IVUS run, a series of IVUS images, or a frame or frames of IVUS images as well as to receive or receive and send signals between an external imaging system (e.g., an angiographic imaging system, extravascular imaging system 404, or the like).

Memory 506 can include instructions 514. During operation processor 504 can execute instructions 514 to configure or cause computing device 502 to co-register images as described herein. For example, processor 504 can execute instructions 514 to receive External images 518 from IVUS imaging system 402 and to receive external images 518 from extravascular imaging system 404. Further, processor 504 can execute instructions 514 to generate graphical components 520 related to the described co-registration procedures and generate (or render) GUI 530 from graphical components 520 and display GUI 530 on display 434. This will be described in greater detail below. However, in general, the co-registration includes receiving indications of IVUS run start location 522 and IVUS run end location 524, generating co-registered pullback path 526 and co-registered side branches 528 and optionally receiving modifications or updates to co-registered pullback path 526 and co-registered side branches 528. It is noted that the pullback path 526 is sometimes referred to as the center line.

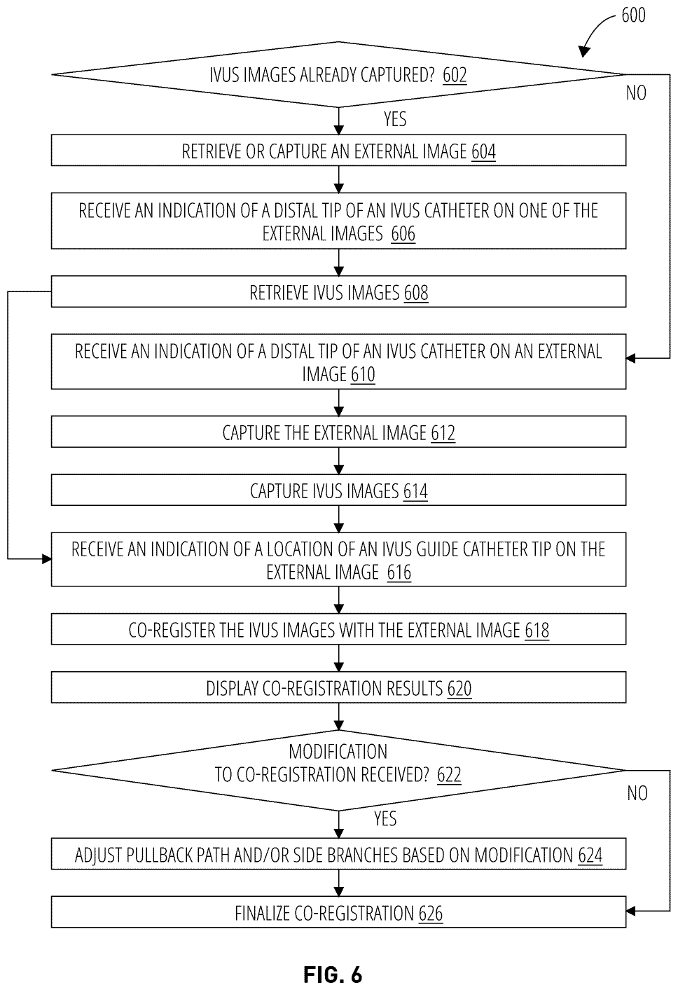

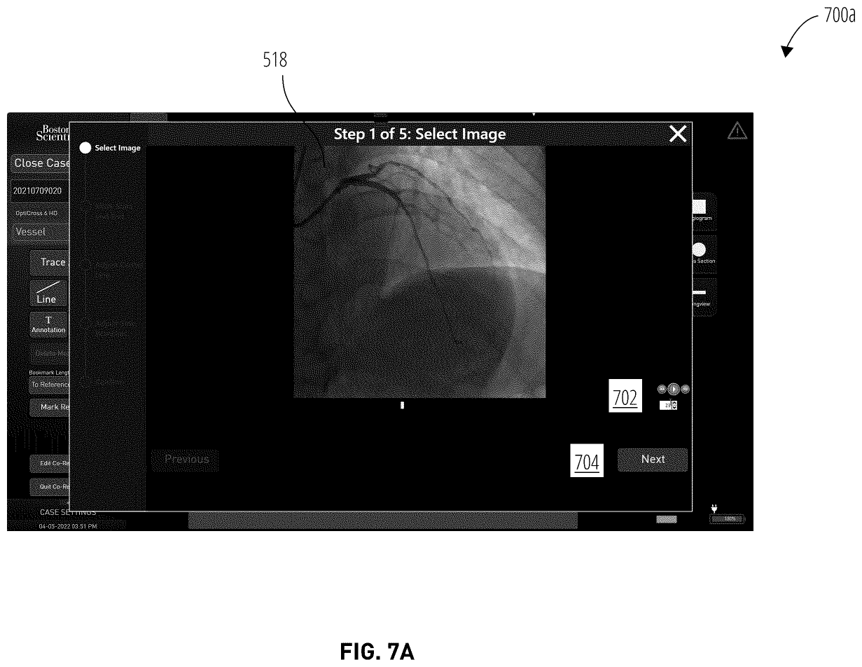

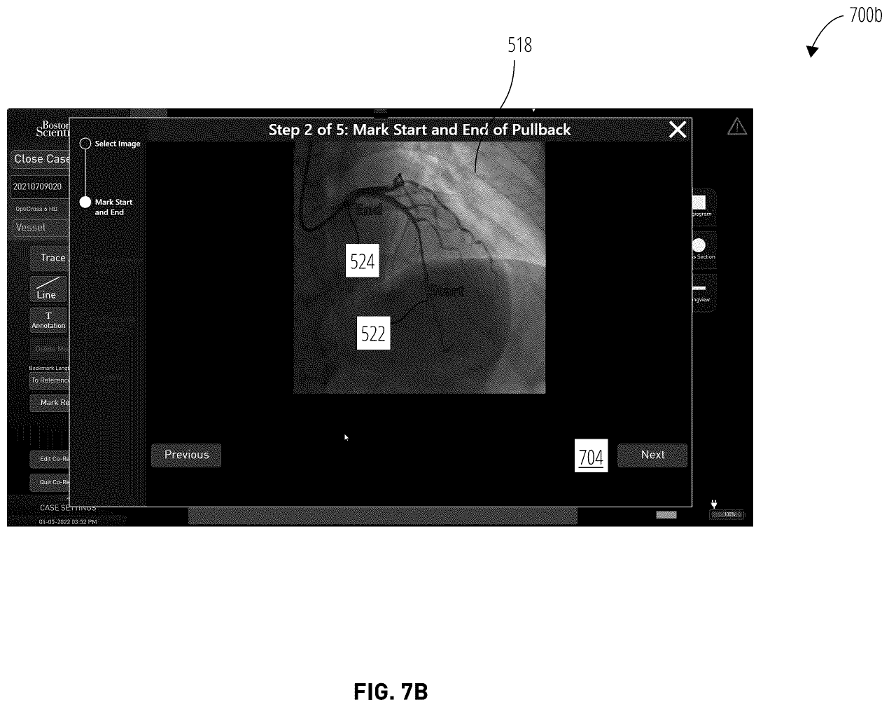

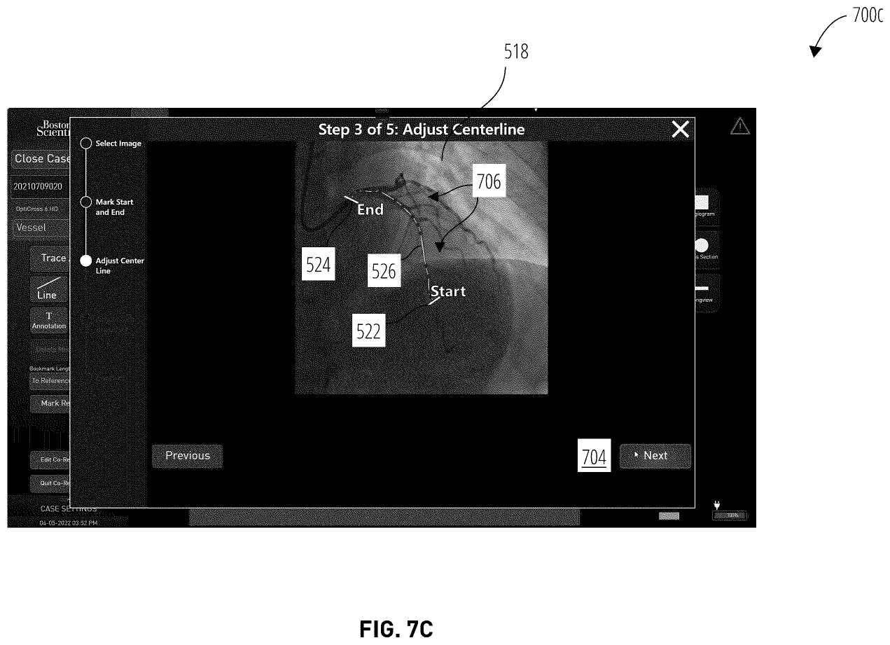

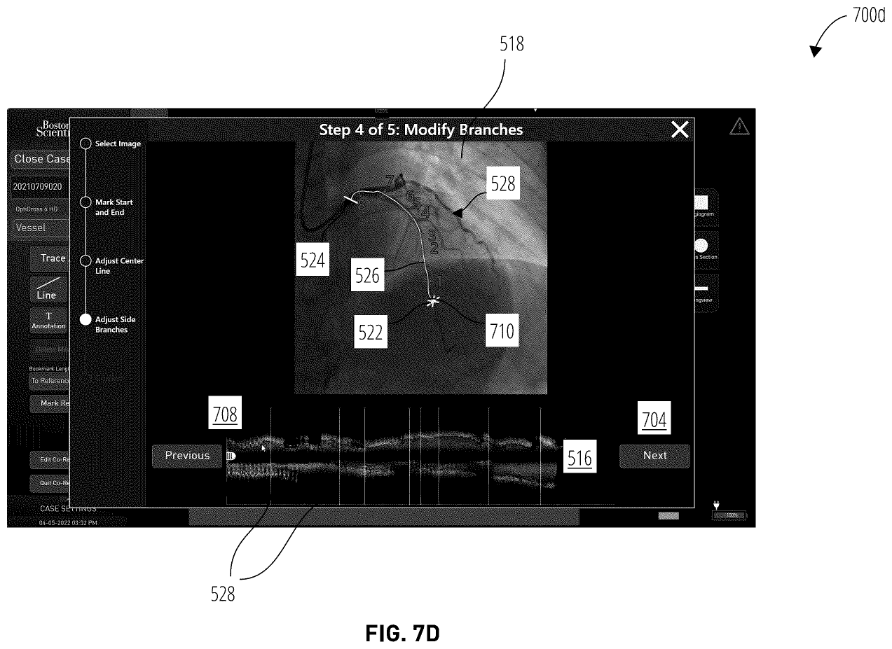

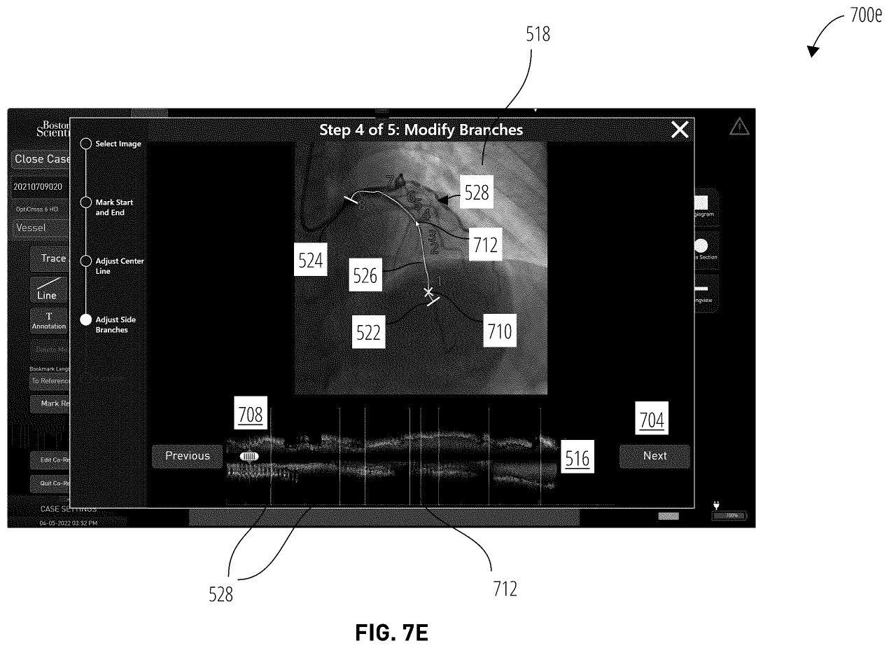

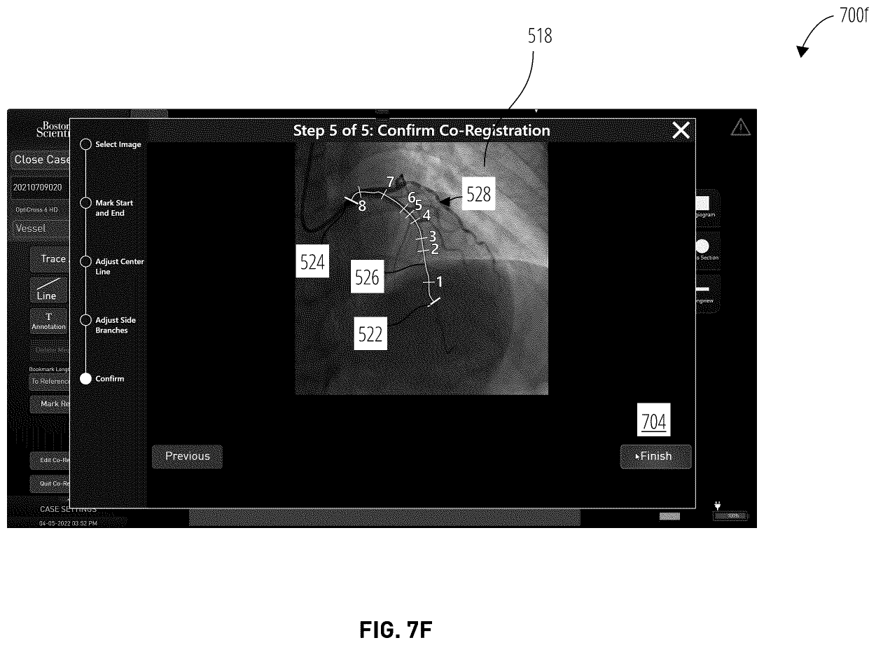

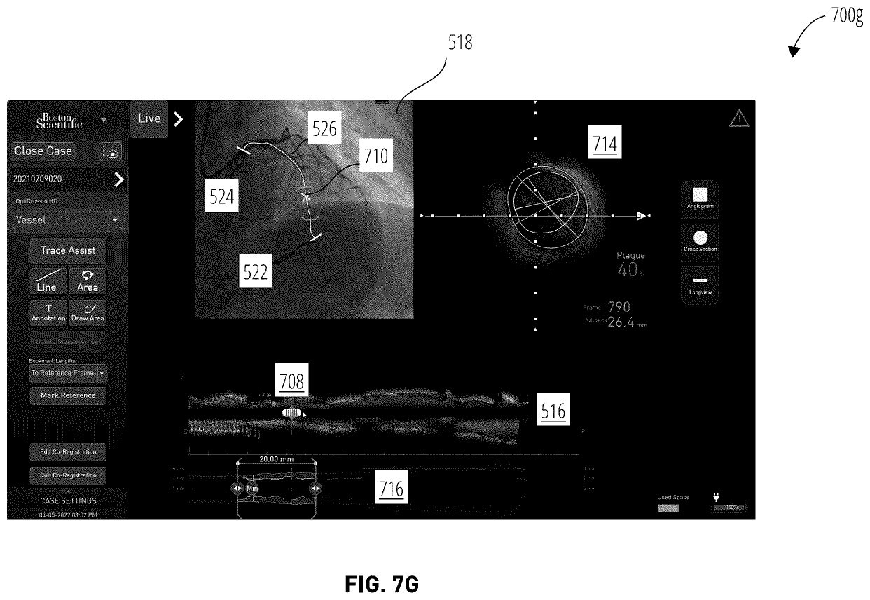

FIG. 6 illustrates the logic flow 600, which can be implemented to co-register IVUS images (e.g., External images 518) with an external image (e.g., one of external images 518). The logic flow 600 can be implemented by co-registration system 500 and will be described with reference to co-registration system 500 for clarity of presentation. However, it is noted that logic flow 600 could also be implemented by an IVUS co-registration system different than co-registration system 500. Further, several examples of a graphical user interface (GUI) and interactions with the GUI are referenced in describing logic flow 600. Examples of such GUIs are provided in FIG. 7A through FIG. 7G. These example GUIs are used to describe logic flow 600. However, logic flow 600 could be implemented using different GUIs that those presented here.

Logic flow 600 can begin at decision block 602. At decision block 602 “IVUS images already captured?” a determination of whether IVUS images have already been captured in made. For example, processor 504 can execute instructions 514 to determine whether External images 518 is already captured or not. From decision block 602, logic flow 600 can continue to block 604 or proceed to block 610. Logic flow 600 can continue from decision block 602 to block 604 based on a determination at decision block 602 that the IVUS images are already captured while logic flow 600 can proceed from decision block 602 to block 610 based on a determination at decision block 602 that the IVUS images have not been captured.

At block 604 “retrieve or capture an external image” an external image can be selected or captured. For example, where several external images are already captured, a one of the external images can be selected. Alternatively, an external image can be captured. For example, often external images (e.g., an angiogram) is already available for the vessel within which the IVUS images were captured. As such, frame of the angiogram can be captured. Processor 504 can execute instructions 514 to generate a GUI to present the external images 518 and to receive an indication of a selection of a frame of the external images 518.

FIG. 7A illustrates GUI 700a, which can be generated as GUI 530. GUI 700a displays an angiogram within which a frame of the angiogram can be selected. In some embodiments, processor 504 can execute instructions 514 to generate GUI 700a and display GUI 700a on display 434. GUI 700a comprises graphical elements or graphical components 520 comprising indications external images 518 (e.g., an angiogram), navigation bar 702 to navigate through external images 518, and selection button 704 to select a frame of external images 518. Processor 504 can execute instructions 514 to receive indications, via I/O devices 508 to navigate through external images 518 and to select a one of external images 518. With some embodiments, the external images 518 are not previously captured or stored. In such an example, processor 504 can execute instructions 514 to receive a live external image (e.g., from extravascular imaging system 404, or the like) and select, capture, or acquire the live image.

Continuing to block 606 “receive an indication of a distal tip of an IVUS catheter on the external image” an indication of a location of a distal tip of an IVUS catheter on the external image is received. Said differently, a location or mark of the start of the IVUS pullback on the external image is received. Processor 504 can execute instructions 514 to receive the indication of the location of the IVUS catheter distal tip on the selected frame of the external images 518 and store the indication as IVUS run start location 522.

Continuing to block 608 “retrieve the IVUS images” IVUS images are retrieved. Processor 504 can execute instructions 514 to retrieve External images 518 (e.g., from memory 506, from an external storage device, or the like). Logic flow 600 continues to block 616 from block 608. However, as noted above, logic flow 600 can proceed from decision block 602 to block 610, for example, where IVUS images are not already captured before co-registration is initiated. At block 610 “receive an indication of a distal tip of an IVUS catheter on an external image” a location of a distal tip of an IVUS catheter on an external image is received. Processor 504 can execute instructions 514 to receive the indication of the location of the IVUS catheter distal tip on the external image and store the indication as IVUS run start location 522. In some embodiments, the indication of the IVUS run start location 522 is received from a user (e.g., via I/O devices 508). In other embodiments, the IVUS run start location 522 is automatically, detected.

Continuing to block 612 “capture the external image” the external image is captured. Processor 504 can execute instructions 514 to capture the external image (e.g., via IVUS/Angio imaging system acquisition circuitries 512 and extravascular imaging system 404, or the like). Further, processor 504 can execute instructions 514 to store the captured external image as external images 518 in memory 506. Continuing to block 614 “capture the IVUS images” the IVUS images are captured. Processor 504 can execute instructions 514 to capture the External images 518 (e.g., via IVUS/Angio imaging system acquisition circuitries 512 and IVUS imaging system 402, or the like). For example, processor 504 can execute instructions 514 to send instructions to start an automatic pullback IVUS image capture process to IVUS imaging system 402. Further, processor 504 can execute instructions 514 to store the captured IVUS images as External images 518 in memory 506.

At block 616 “receive an indication of a location of an IVUS guide catheter tip on the external image” an indication of a location of a distal tip of an IVUS guide catheter on the external image is received. Said differently, a location or mark of the end of the IVUS pullback on the external image is received. Processor 504 can execute instructions 514 to receive the indication of the location of the IVUS guide catheter distal tip on the selected (or captured) frame of the external images 518 and store the indication as IVUS run end location 524. In some embodiments, the indication of the IVUS run end location 524 is received from a user (e.g., via I/O devices 508). In other embodiments, the IVUS run end location 524 is automatically, detected.

FIG. 7B illustrates GUI 700b, which can be generated as GUI 530. GUI 700b displays a frame of an angiogram (e.g., a frame of external images 518) within which the location of the distal tip of the IVUS catheter and the distal tip of the IVUS guide catheter can be marked. In some embodiments, processor 504 can execute instructions 514 to generate GUI 700b and display GUI 700b on display 434. GUI 700b comprises graphical elements or graphical components 520 comprising a frame of external images 518, selection button 704, and IVUS run start location 522 and IVUS run end location 524. Processor 504 can execute instructions 514 to receive indications, via I/O devices 508, and to generate graphical components 520 to display graphical representations of the indications on GUI 700b relative to the frame of the external images 518. For example, processor 504 can execute instructions 514 to display IVUS run start location 522 corresponding to the indication of the location of the IVUS catheter distal tip or the start of the IVUS pullback and display IVUS run end location 524 corresponding to the indication of the location of the IVUS guide catheter or the end of the IVUS pullback.

Continuing to block 618 “co-register the IVUS images with the external image” the IVUS images are co-registered with the external image. Frames of the External images 518 are mapped to or registered onto locations along the vessel 202 depicted in the frame of external images 518. Processor 504 can execute instructions 514 to co-register the images. In some examples, processor 504 can co-register the images using a machine learning model trained to co-register IVUS images to an external image. Processor 504 can execute instructions 514 to identify a center line of the vessel 202 corresponding to the pullback path of the IVUS catheter through the vessel on external images 518 and to register side branches along the longitudinal length of the vessel 202 and store the results as co-registered pullback path 526 and co-registered side branches 528.

Continuing to block 620 “display editable co-registration results” the co-registration results can be displayed in editable or manipulatable format. Processor 504 can execute instructions 514 to generate graphical components 520 comprising indications of the co-registration results (e.g., indications of co-registered pullback path 526 and co-registered side branches 528). Processor 504 can execute instructions 514 to generate GUI 530 from graphical components 520 and display GUI 530 on display 434 where the results can be manipulated by a user.

Continuing to decision block 622 “modifications to co-registration results received?” a determination whether modifications to the co-registration results are received is made. Processor 504 can execute instructions 514 to determine whether modifications to co-registered pullback path 526 and/or co-registered side branches 528 are received. In some examples, the modifications are received via the graphical components GUI 530 (e.g., via touch screen, via a mouse, or the like). Logic flow 600 can continue from decision block 622 to block 624 or proceed from decision block 622 to block 626. For example, logic flow 600 can continue from decision block 622 to block 624 based on a determination at decision block 622 that modification(s) are received while decision block 622 can proceed from decision block 622 to block 626 based on a determination at decision block 622 that modification are not received. With some examples, processor 504 can execute instructions 514 to receive an indication that selection button 704 is activated indicating that modifications are not received.

At block 624 “adjust pullback path and/or side branches based on modifications” the pullback path and/or side branches can be adjusted based on modifications as outlined below.

FIG. 7C illustrates GUI 700c, which can be generated as GUI 530. GUI 700c displays the frame of the angiogram (e.g., the frame of external images 518 selected or captured as outlined above) with the results of the co-registration displayed. Processor 504 can execute instructions 514 to generate GUI 700c and display GUI 700c on display 434. GUI 700c comprises graphical elements or graphical components 520 comprising the frame of external images 518, selection button 704, and IVUS run start location 522, IVUS run end location 524, co-registered pullback path 526 and co-registered side branches 528.

As noted, at least some of the graphical components 520 of GUI 700c are manipulatable. That is, a user can move the graphical components to change locations represented by the graphical components. For example, in GUI 700c, co-registered pullback path 526 is adjustable. Processor 504 can execute instructions 514 to receive indications of manipulations to the co-registration results and update the co-registration results based on the received manipulations. For example, processor 504 can execute instructions 514 to receive, via I/O devices 508, adjustments or manipulations to the adjustment nodes 706 of co-registered pullback path 526 and save the manipulations in memory 506 as co-registered pullback path 526. Further, processor 504 can execute instructions 514 to update the GUI 530 (e.g., GUI 700c, or the like) in real time to represent the adjusted co-registration results (e.g., co-registered pullback path 526).

FIG. 7D illustrates GUI 700d showing manipulation of the co-registered side branches 528. Processor 504 can execute instructions 514 to generate GUI 700d and display GUI 700d on display 434. GUI 700d comprises graphical elements or graphical components 520 comprising the frame of external images 518, selection button 704, IVUS run start location 522, IVUS run end location 524, co-registered pullback path 526, co-registered side branches 528 represented on the external images 518, IVUS images 516 and a slider 708 to move along the longitudinal length of the IVUS images 516 and a slider marker 710 showing the position of the slider 708 relative to the location along the longitudinal length of the vessel 202 in the frame of external images 518.

Like GUI 700c, at least some of the graphical components 520 of GUI 700d are manipulatable. That is, a user can move the graphical components to change locations represented by the graphical components. For example, in GUI 700d, co-registered side branches 528 are adjustable. Processor 504 can execute instructions 514 to receive indications of manipulations to the co-registration results and update the co-registration results based on the received manipulations. For example, processor 504 can execute instructions 514 to receive, via I/O devices 508, adjustments or manipulations to the co-registered side branches 528 and save the manipulations in memory 506 as co-registered side branches 528. Further, processor 504 can execute instructions 514 to update the GUI 530 in real time to represent the adjusted co-registration results (e.g., co-registered pullback path 526). Further, slider 708 can be manipulated to move the slider marker 710 along the length of vessel 202 shown in the frame of the external images 518 while the slider 708 moves along the length of IVUS images 516 to visually match the co-registered side branches 528 between the IVUS images 516 and external images 518.

FIG. 7E illustrates GUI 700e, which is like GUI 700d with “real-time” adjustments to co-registered side branches 528 reflected in the GUI 700e. For example, processor 504 can execute instructions 514 to receive, via I/O devices 508, an indication to move one (e.g., branch number 4) of co-registered side branches 528. In some examples, only the selected one of co-registered side branches 528 is adjusted. In other examples, as the selected branch is moved, other ones of the co-registered side branches 528 may also move. For example, all upstream (or proximal to the moved branch) can be adjusted similarly, all downstream (or distal to the moved branch (can be adjusted similarly, or all branches can be adjusted. In the case illustrated in FIG. 7D and FIG. 7E, where co-registered side branches 528 number 4 is adjusted, co-registered side branches 528 numbers 5, 6, 7, and 8 will also be adjusted.

At block 626 “finalize co-registration” the co-registration can be finalized. Processor 504 can execute instructions 514 to generate graphical components 520 from IVUS run start location 522, IVUS run end location 524, co-registered pullback path 526, and co-registered side branches 528 and GUI 530 from graphical components 520 to display graphical representations of the co-registration. FIG. 7F illustrates GUI 700f displaying graphical components 520 comprising IVUS run start location 522, IVUS run end location 524, co-registered pullback path 526, co-registered side branches 528 represented on the frame of external images 518. Processor 504 can execute instructions 514 to receive an indication, via I/O devices 508, that the represented co-registration (e.g., IVUS run start location 522, IVUS run end location 524, co-registered pullback path 526, and external images 518) is complete. For example, processor 504 can receive an indication that the selection button 704 is activated (e.g., clicked) confirming the displayed co-registration. Further, although not described above, return or “previous” buttons can be present in the GUIs (e.g., GUI 700f) to return to a prior step of adjusting co-registration, or the like.

Block 626 can further include generating a GUI to display the results of the co-registration. FIG. 7G illustrates GUI 700g. GUI 700g can be generated as GUI 530. Processor 504 can execute instructions 514 to generate GUI 530 comprising graphical components 520 like depicted in GUI 700g. GUI 700g comprises graphical representations of IVUS images 516 and a vessel profile 716 of the vessel and lumen borders of vessel 202 represented in IVUS images 516. GUI 700g further includes slider 708 to move longitudinally along IVUS images 516 and corresponding slider marker 710 representing the location of slider 708 on external images 518. Additionally, GUI 700g includes a cross section view 714 of the vessel (or a frame of the IVUS images 516) at the location of slider 708 and vessel and lumen borders.

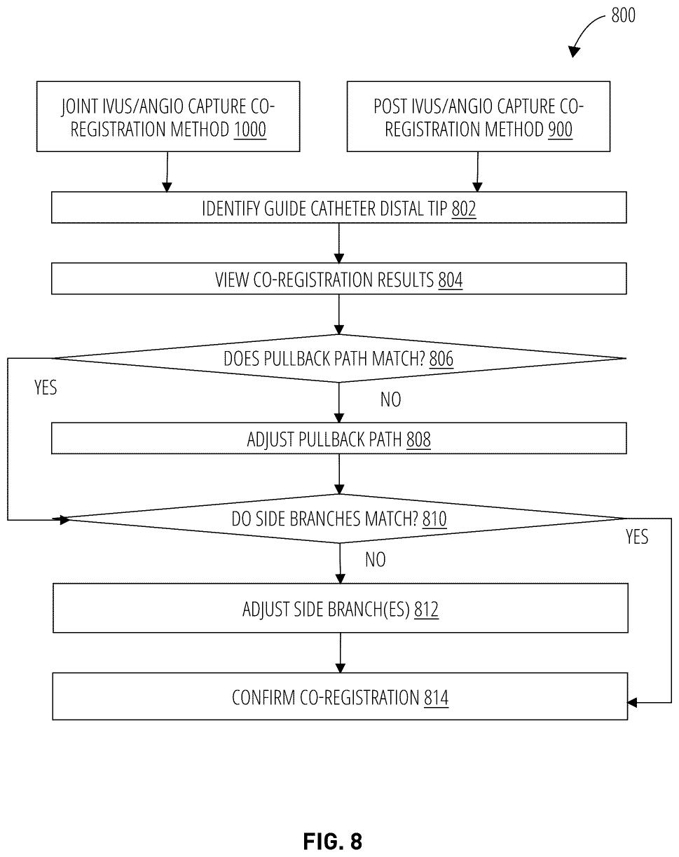

As noted, the present disclosure provides a workflow for a physician in which co-registration can be done either before image acquisition or after. FIG. 8 illustrates a method 800 to co-register IVUS images and an external image. Method 800 is described with reference to the tools, systems, and GUIs described above. Method 800 can begin with either method 1000 or method 900. In particular, the workflow is different depending upon whether IVUS images are already captured or not. For example, where IVUS images are already captured, method 800 can start with method 900 while where co-registration is done or initiated before IVUS images are captured method 800 can begin at method 1000.

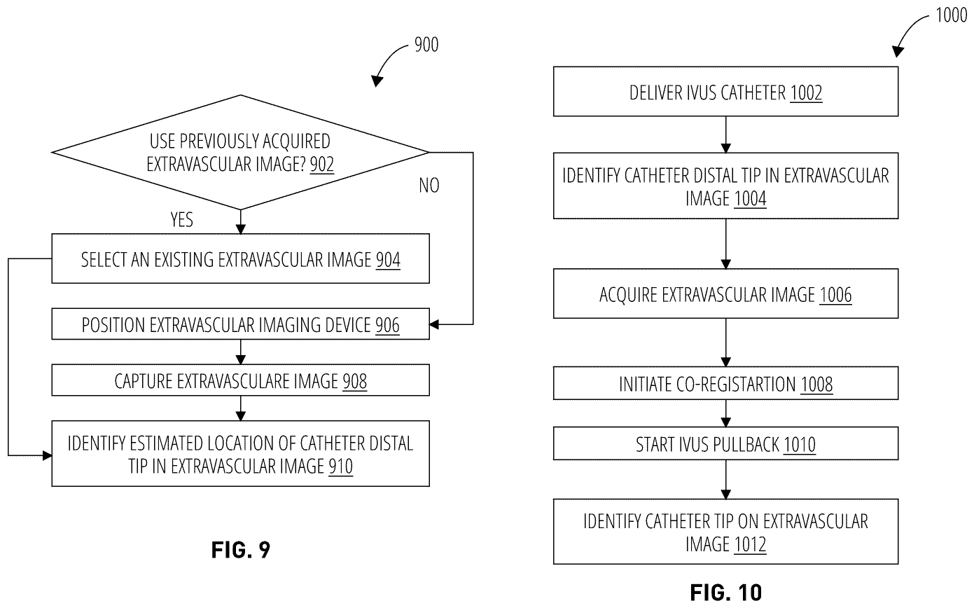

FIG. 9 illustrates the method 900. The method 900 can begin at decision block 902 “use previously acquired extravascular image?” the physician can determine whether to use the previously acquired extravascular image. Method 900 can continue from decision block 902 to block 904 based on a determination to use previously acquired extravascular images while method 900 can proceed from decision block 902 to block 906 based on a determination not to use previously acquired extravascular images (either because they don't exist of the physician wants to capture another).

At block 904 “select an existing extravascular image” an existing extravascular image can be selected. For example, a physician can select a frame of external images 518 via GUI 700a, or the like. At block 906 “position extravascular imaging device” extravascular imaging system 404 can be positioned over patient 412 to capture external images 518 and at block 908, the external images 518 is captured.

Method 900 can continue from both block 904 and block 908 to block 910. At block 910 “identify estimated location of the catheter distal tip in extravascular image” the physician can identify the estimated location of the start of the IVUS pullback (e.g., most distal location of the IVUS catheter 102 in vessel 202, or the like). For example, the physician can identify the IVUS run start location 522 via GUI 700b.

FIG. 10 illustrates the method 1000. The method 1000 can begin at block 1002 “deliver IVUS catheter” the physician can deliver or place the IVUS imaging catheter 420 of IVUS imaging system 402 in patient 412. Continuing to block 1004 “identify catheter distal tip in extravascular image” the distal tip of the IVUS imaging catheter 420 can be identified in an extravascular image (e.g., via extravascular imaging system 404, or the like). Continuing to block 1006 “acquire extravascular image” the extravascular image can be acquired (e.g., via extravascular imaging system 404 and computing device 406, or the like). Continuing to block 1008 initiate co-registration” co-registration can be initiated (e.g., via computing device 406). Continuing to block 1010 “start IVUS pullback” the IVUS pullback or IVUS image acquisition can be initiated (e.g., via IVUS imaging system 402 and computing device 406).

Continuing to block 1012 “identify catheter tip in extravascular image” the physician can identify the location of the start of the IVUS pullback (e.g., most distal location of the IVUS catheter 102 in vessel 202, or the like). For example, the physician can identify the IVUS run start location 522 via GUI 700b.

Returning to method 800 and FIG. 8. The method 800 can continue from both method 900 and method 1000 to block 802. At block 802 “identify guide catheter distal tip” the guide catheter distal tip can be identified. For example, the physician can identify the IVUS run end location 524 via GUI 700b.

Continuing to block 804 “view co-registration results” the co-registration results are views (e.g., via GUI 530). Continuing to decision block 806 “does pullback path match?” a determination whether the pullback path matches is made. For example, the physician can determine whether the co-registered pullback path 526 matches the vessel 202 represented in the frame of external images 518. Method 800 can continue from decision block 806 to block 808 based on a determination that the pullback path does not match while method 800 can proceed from decision block 806 to decision block 810 based on a determination that the pullback path does match. At block 808 “adjust the pullback path” the pullback path can be adjusted. For example, the physician can adjust the co-registered pullback path 526 via GUI 700c.

Method 800 continues from block 808 to decision block 810. At decision block 810 “do side branches match?” a determination whether the side branches match is made. For example, the physician can determine whether the co-registered side branches 528 match the vessel 202 represented in the frame of external images 518. Method 800 can continue from decision block 810 to block 812 based on a determination that the side branches does not match while method 800 can proceed from decision block 810 to block 814 based on a determination that the side branches do match. At block 812 “adjust side branch(es)” a side branch or multiple side branches can be adjusted. For example, the physician can adjust the co-registered side branches 528 via GUI 700d and/or GUI 700e.

At block 814 “confirm the co-registration” the co-registration can be confirmed. For example, a physician can confirm the co-registration via GUI 700f and subsequently view the results view GUI 700g.

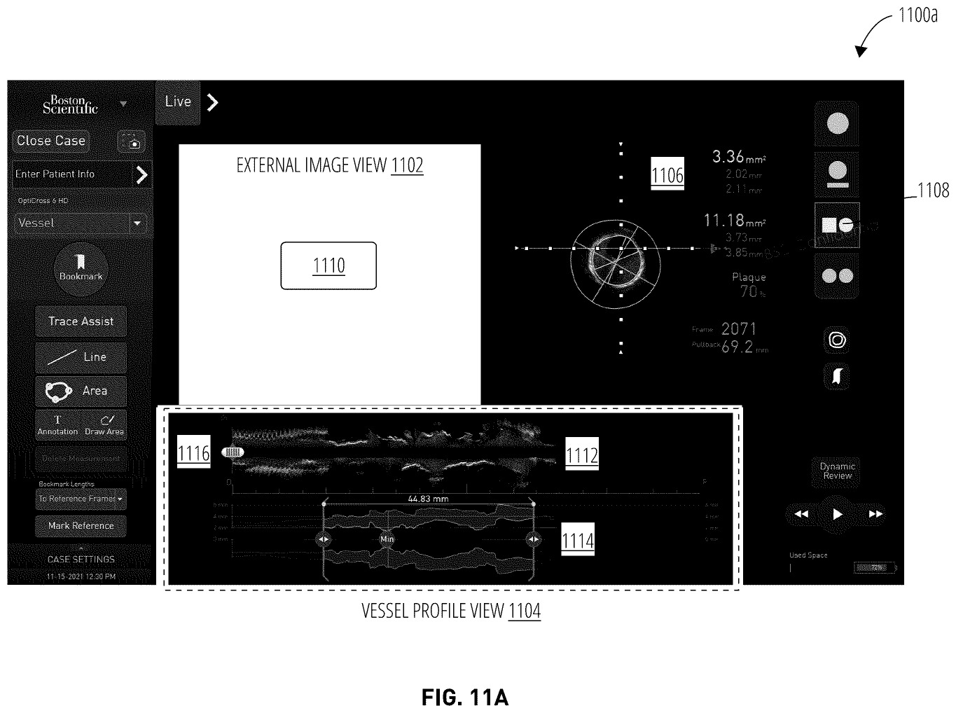

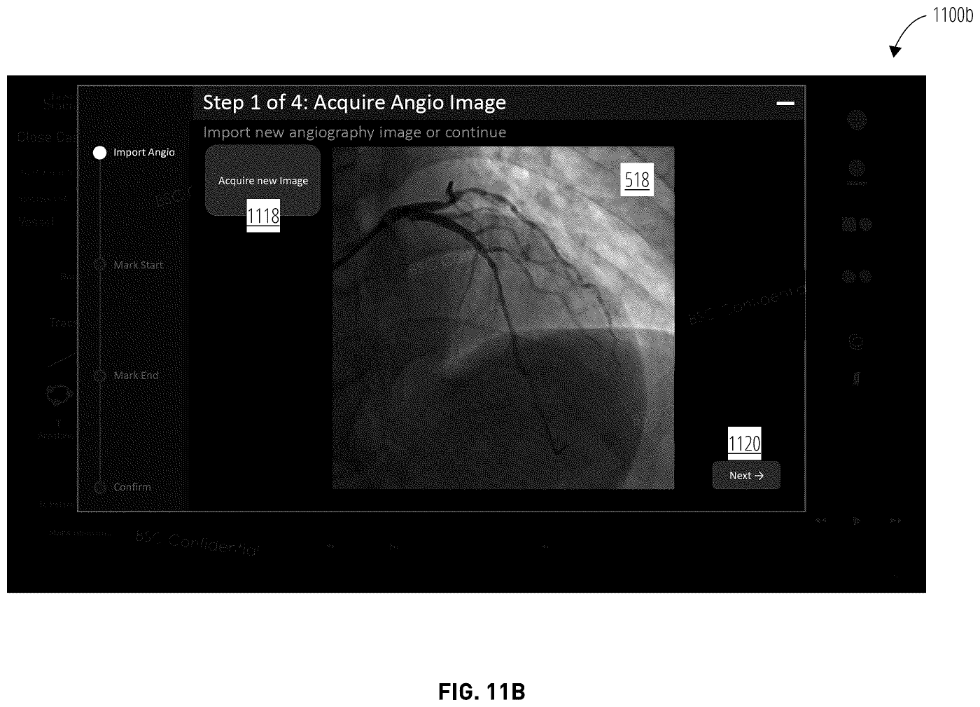

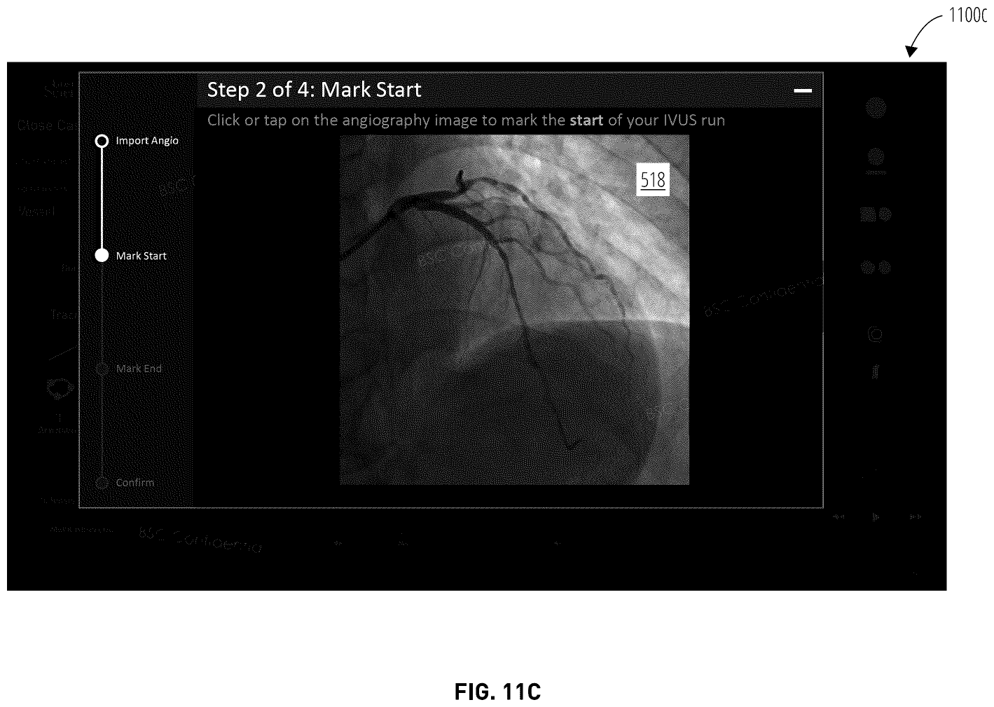

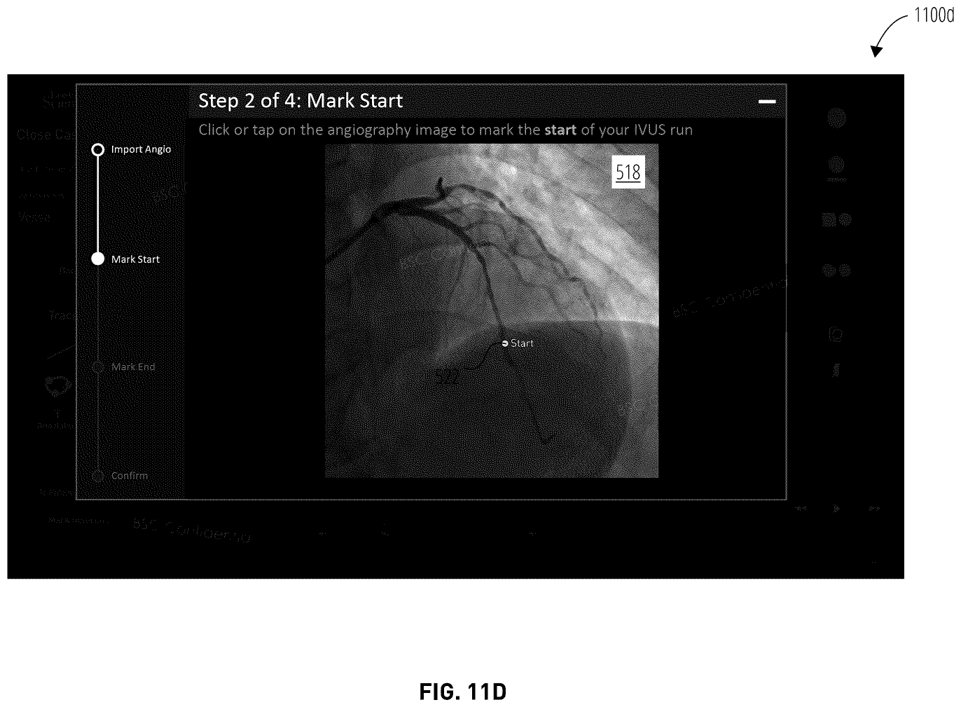

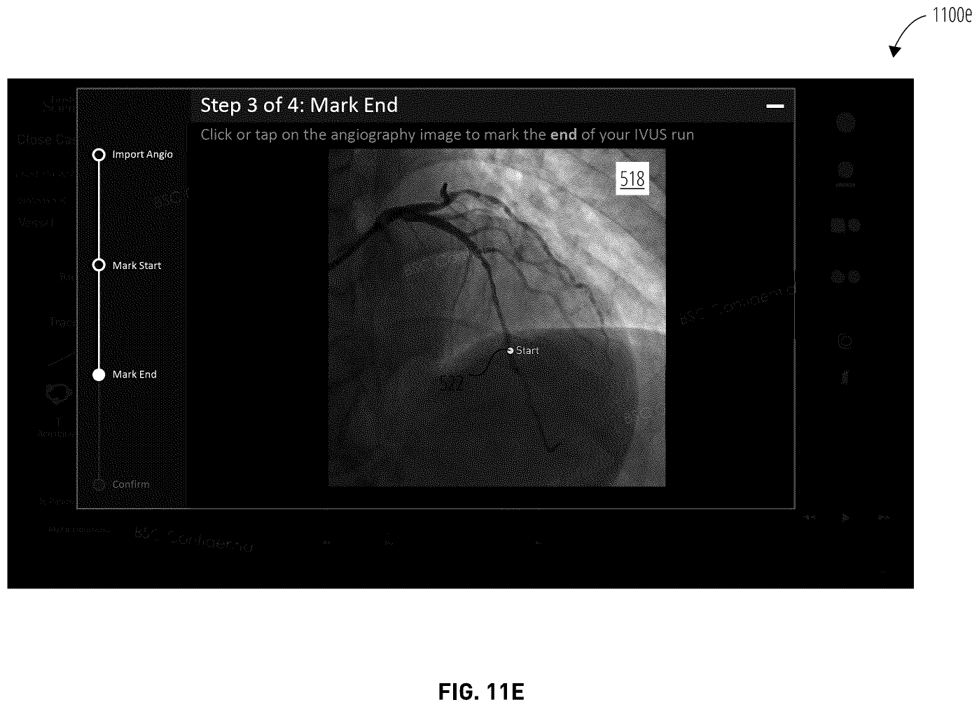

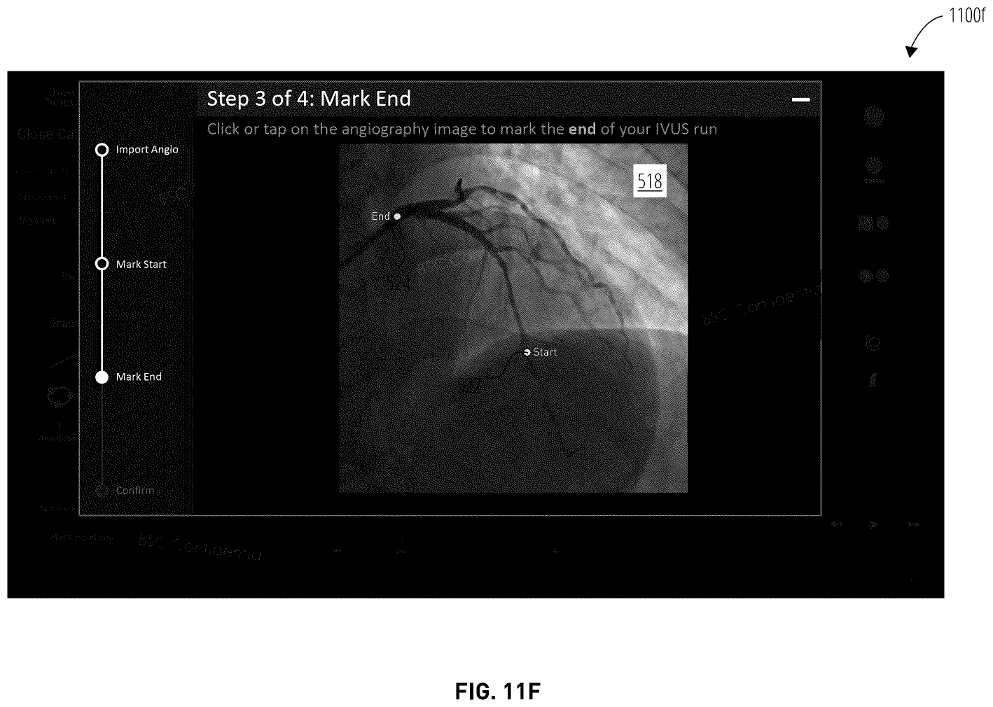

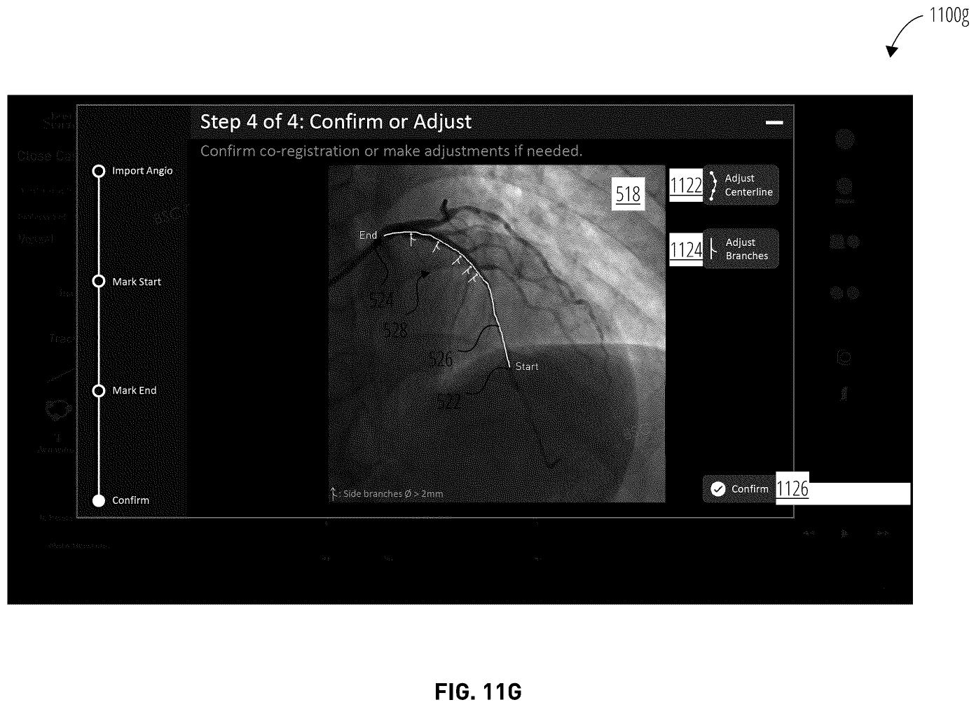

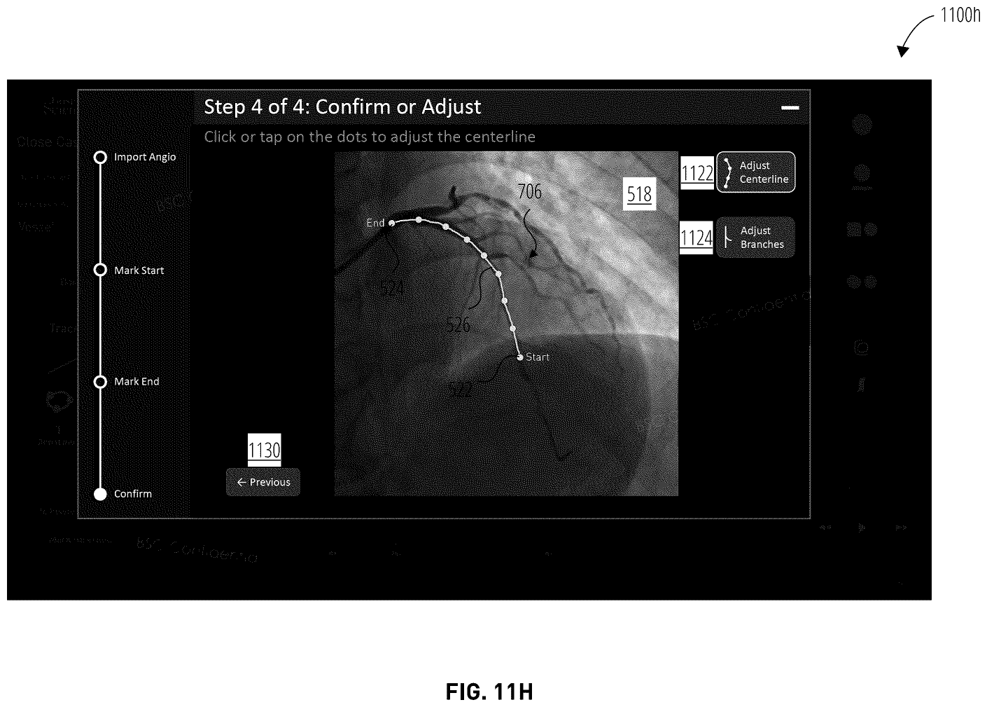

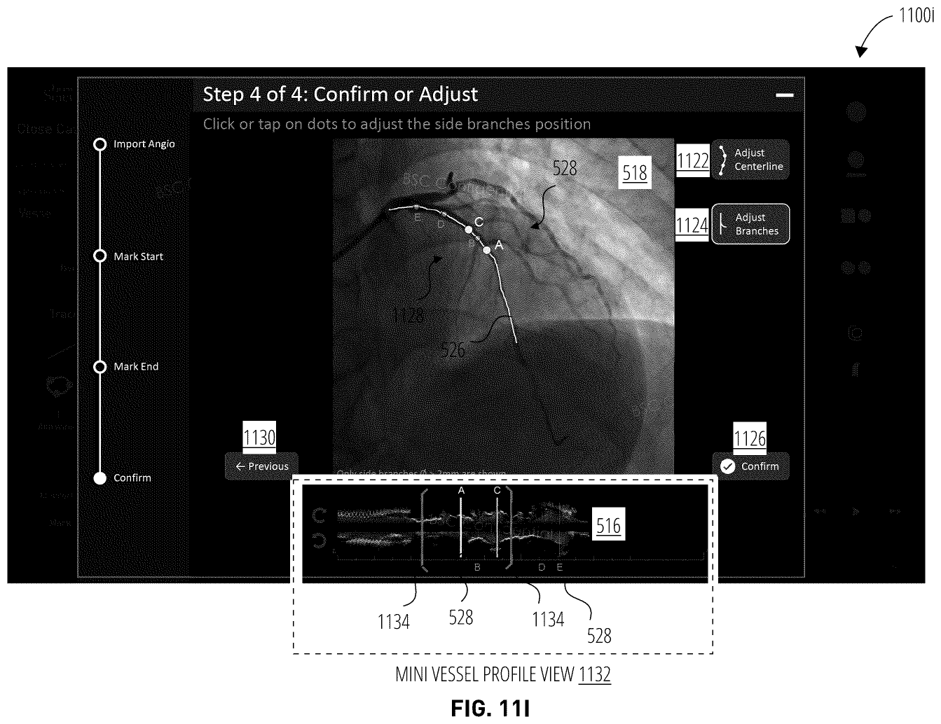

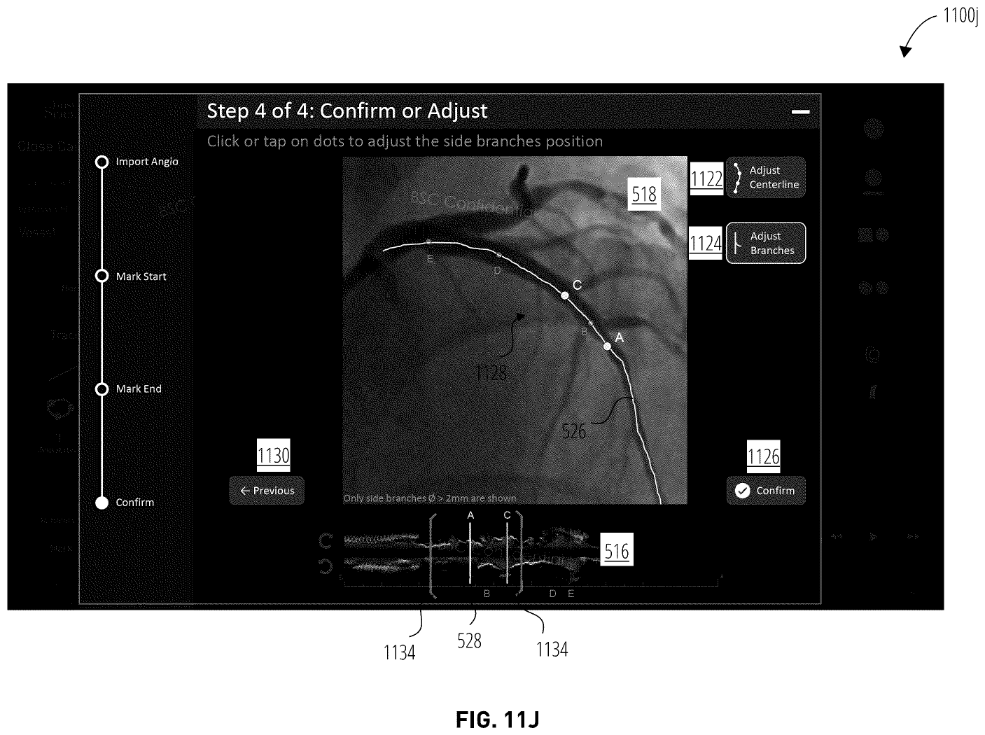

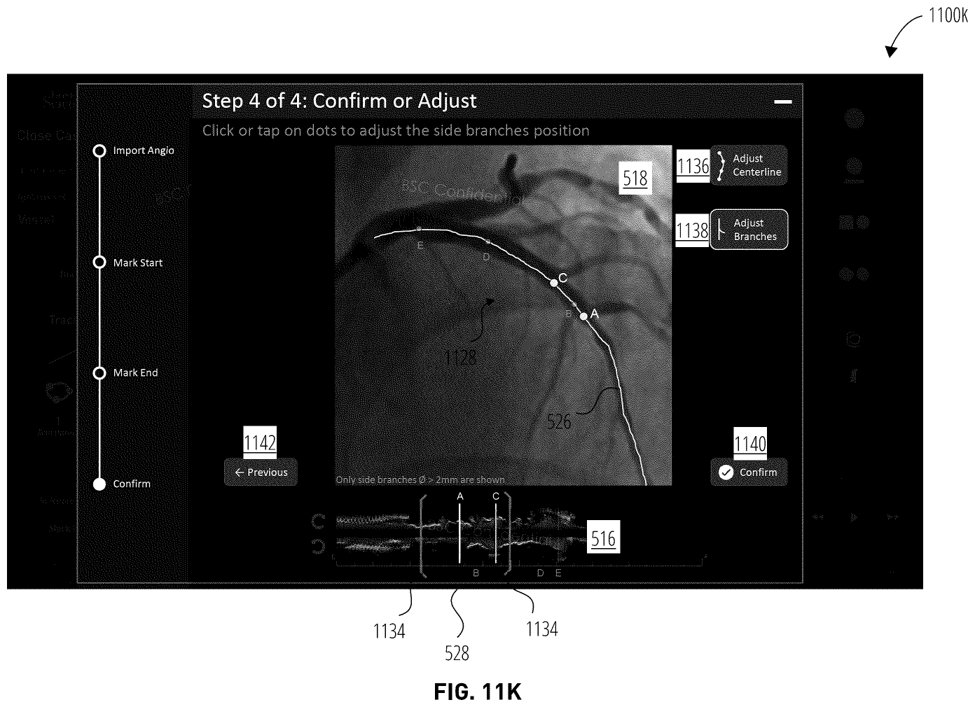

FIG. 11A illustrates GUI 1100a. GUI 1100a can be generated as GUI 530. Processor 504 can execute instructions 514 to generate GUI 530 comprising graphical components 520. GUI 1100a comprises an external image view 1102, cross section view 1106, vessel profile view 1104, and various menus and selection buttons. For example, GUI 1100a includes co-registration view button 1108 to select a co-registration view like shown in this figure. The external image view 1102 includes external image acquisition button 1110 to select and/or acquire an external image (e.g., external images 518) as described above. With some examples, processor 504 can execute instructions 514 to capture and/or select a single frame from a live angiographic feed while in other examples processor 504 can execute instructions 514 to capture an angiogram and select a frame from the angiogram (e.g., as described previously). In further examples, processor 504 can execute instructions 514 to recapture (or repeatedly capture) a frame of a live external image (e.g., angiogram, or the like).

The vessel profile view 1104 includes a graphical representation of the vessel border and lumen border and as well as a longitudinal view 1112 of the vessel based on the frames of IVUS images 516. Further, vessel profile view 1104 includes a graphical depiction of the borders of the vessel and lumen represented in IVUS images 516 (e.g., vessel/lumen border 1114) as well as a slider 1116 to pan or traverse through the frames of the IVUS images 516.

The cross section view 1106 includes a cross-section view of the vessel (e.g., a frame of the IVUS images 516) as well as cross-section views of the borders (e.g., vessel/lumen border 1114) at the location of slider 1116.

FIG. 11B illustrates GUI 1100b, which can be generated as GUI 530. GUI 1100b can be displayed when a user selects co-registration view button 1108 and external image acquisition button 1110 to co-register one of external images 518 with frames of IVUS images 516 as described herein. GUI 1100b comprises graphical elements or graphical components 520 comprising indications external images 518 (e.g., an angiogram), acquire new image button 1118 to acquire a new one of external images 518 and proceed button 1120 to continue to co-registration. Processor 504 can execute instructions 514 to receive indications, via I/O devices 508 to select one of external images 518 and to start co-registration using the selected image.

FIG. 11C illustrates GUI 1100c, which can be generated as GUI 530. GUI 1100c displays the frame of the angiogram (e.g., the frame of external images 518 selected or captured as outlined above via GUI 1100b). Processor 504 can execute instructions 514 to generate GUI 1100c and display GUI 1100c on display 434. GUI 1100c comprises graphical elements or graphical components 520 comprising the frame of external images 518 and can be interacted with (e.g., via I/O devices 508, or the like) to select IVUS run start location 522.

FIG. 11D illustrates GUI 1100d, which can be generated as GUI 530. GUI 1100d displays the frame of the angiogram (e.g., the frame of external images 518 selected or captured as outlined above via GUI 1100b). Processor 504 can execute instructions 514 to generate GUI 1100d and display GUI 1100d on display 434. GUI 1100d comprises graphical elements or graphical components 520 comprising the frame of external images 518 and can be interacted with (e.g., via I/O devices 508, or the like) to select IVUS run start location 522. For example, processor 504 can execute instructions 514 to add IVUS run start location 522 to a location on external images 518 based on a touch, mouse, or other input or gesture received via display 434 or I/O devices 508.

FIG. 11E illustrates GUI 1100e, which can be generated as GUI 530. GUI 1100e displays the frame of the angiogram (e.g., the frame of external images 518 selected or captured as outlined above via GUI 1100b). Processor 504 can execute instructions 514 to generate GUI 1100e and display GUI 1100e on display 434. GUI 1100e comprises graphical elements or graphical components 520 comprising the frame of external images 518 and can be interacted with (e.g., via I/O devices 508, or the like) to select IVUS run end location 524.

FIG. 11F illustrates GUI 1100f, which can be generated as GUI 530. GUI 1100f displays the frame of the angiogram (e.g., the frame of external images 518 selected or captured as outlined above via GUI 1100b). Processor 504 can execute instructions 514 to generate GUI 1100f and display GUI 1100f on display 434. GUI 1100f comprises graphical elements or graphical components 520 comprising the frame of external images 518 and can be interacted with (e.g., via I/O devices 508, or the like) to select IVUS run start location 522. For example, processor 504 can execute instructions 514 to add IVUS run end location 524 to a location on external images 518 based on a touch, mouse, or other input or gesture received via display 434 or I/O devices 508.

FIG. 11G illustrates GUI 1100g, which can be generated as GUI 530. GUI 1100g displays the frame of the angiogram (e.g., the frame of external images 518 selected or captured as outlined above) with the results of the co-registration displayed. Processor 504 can execute instructions 514 to generate GUI 1100g and display GUI 1100g on display 434. GUI 1100g comprises graphical elements or graphical components 520 comprising the frame of external images 518, adjust pullback path button 1122, adjust branches button 1124, and confirm co-registration button 1126. Further, GUI 1100g comprises indications of IVUS run start location 522, IVUS run end location 524, co-registered pullback path 526 and co-registered side branches 528.

As noted, the present disclosure provides that a user can manipulate or adjust the co-registration results. Processor 504 can execute instructions 514 to receive an indication to enter a pullback path adjustment interface (e.g., via activation of adjust pullback path button 1122) or a side branch adjustment interface (e.g., via activation of adjust branches button 1124) or to confirm the co-registration results (e.g., via activation of confirm co-registration button 1126).

FIG. 11H illustrates GUI 1100h, which can be generated as GUI 530. Processor 504 can execute instructions 514 to generate GUI 1100h and display GUI 1100h on display 434 responsive to activation of the adjust pullback path button 1122. GUI 1100h comprises graphical components 520 depicting the frame of the angiogram (e.g., the frame of external images 518 selected or captured as outlined above) with the co-registered pullback path 526 and adjustment nodes 706. Processor 504 can execute instructions 514 to receive adjustments to the co-registered pullback path 526 via manipulation of adjustment nodes 706 through various input devices (e.g., touch screen display, I/O devices 508, or the like). Further, processor 504 can execute instructions 514 to update the GUI 530 (e.g., GUI 1100h, or the like) in real time to represent the adjusted co-registration results (e.g., co-registered pullback path 526).

FIG. 11I illustrates GUI 1100i, which can be generated as GUI 530. Processor 504 can execute instructions 514 to generate GUI 1100i and display GUI 1100i on display 434 responsive to activation of the adjust branches button 1124. GUI 1100h comprises graphical components 520 depicting the frame of the angiogram (e.g., the frame of external images 518 selected or captured as outlined above) with the co-registered pullback path 526, the co-registered side branches 528, and branch adjustment nodes 1128. Further, GUI 1100h includes return button 1130 to return to the previous GUI (e.g., GUI 1100h, or the like) and confirm co-registration button 1126.

Further, GUI 1100i incudes graphical components 520 depicting a mini vessel profile view 1132 including the longitudinal representation of IVUS images 516 as well as key frames 1134 and co-registered side branches 528. In some embodiments, the co-registered side branches can be depicted as lines, depicted with numbers, depicted with letters, or other indications to differentiate between the different side branches.

Processor 504 can execute instructions 514 to receive adjustments to the co-registered side branches 528 via manipulation of branch adjustment nodes 1128 through various input devices (e.g., touch screen display, I/O devices 508, or the like). Further, processor 504 can execute instructions 514 to update the GUI 530 (e.g., GUI 1100i, or the like) in real time to represent the adjusted co-registration results (e.g., co-registered side branches 528).

In some embodiments, ones of the co-registered side branches 528 can be adjusted by selecting two (or more) side branches and moving one of the selected side branches. In some embodiments, processor 504 can execute instructions 514 to emphasize (e.g., highlight, bold, etc.) the ones of co-registered side branches 528 selected for adjustment via branch adjustment nodes 1128 in the mini vessel profile view 1132. With some embodiments, the selected side branches can be adjusted via interaction with the graphical elements of branch adjustment nodes 1128 depicted on external images 518 or via interaction with the graphical elements of co-registered side branches 528 depicted in mini vessel profile view 1132.

FIG. 11J and FIG. 11K illustrate GUI 1100j and GUI 1100k, respectively. GUI 1100j and GUI 1100k comprise graphical components 520 depicting elements like GUI 1100i and further illustrate adjustments to branch adjustment nodes 1128. For example, these figures depict branch adjustment nodes 1128 corresponding to co-registered side branches 528 “A” and “C” being adjusted. Processor 504 can execute instructions 514 to generate GUI 1100j and/or GUI 1100k responsive to receiving adjustments or manipulations to co-registered side branches 528 (e.g., via a touch screen, via I/O devices 508, or the like).

It is noted that GUIs 1100a through GUI 1100k can be generated as part of logic flow 600. However, further discussion of logic flow 600 is omitted for brevity.

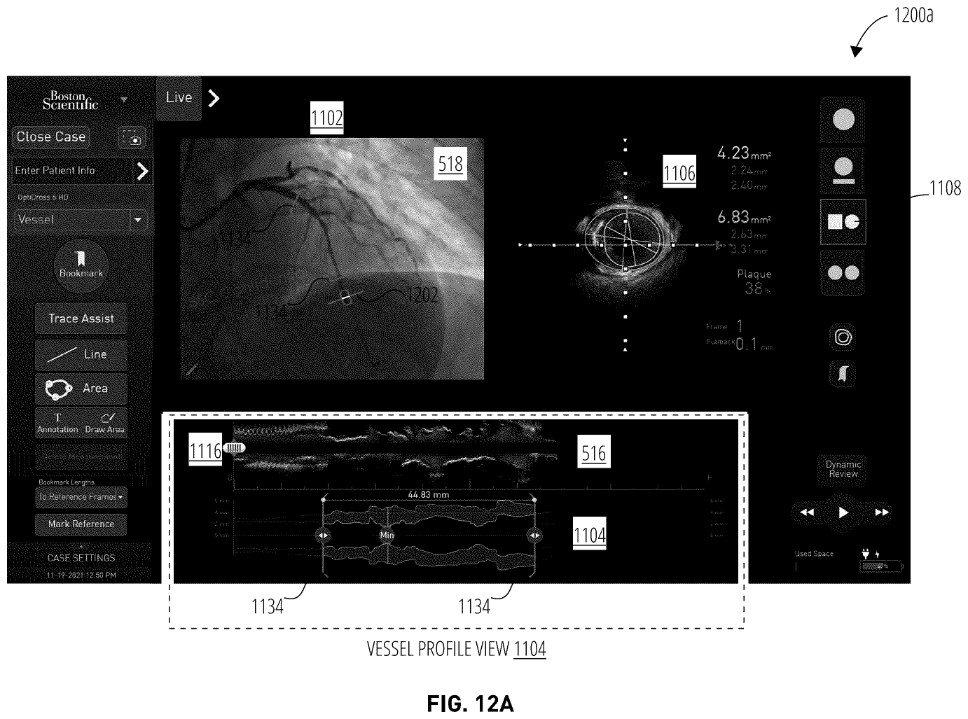

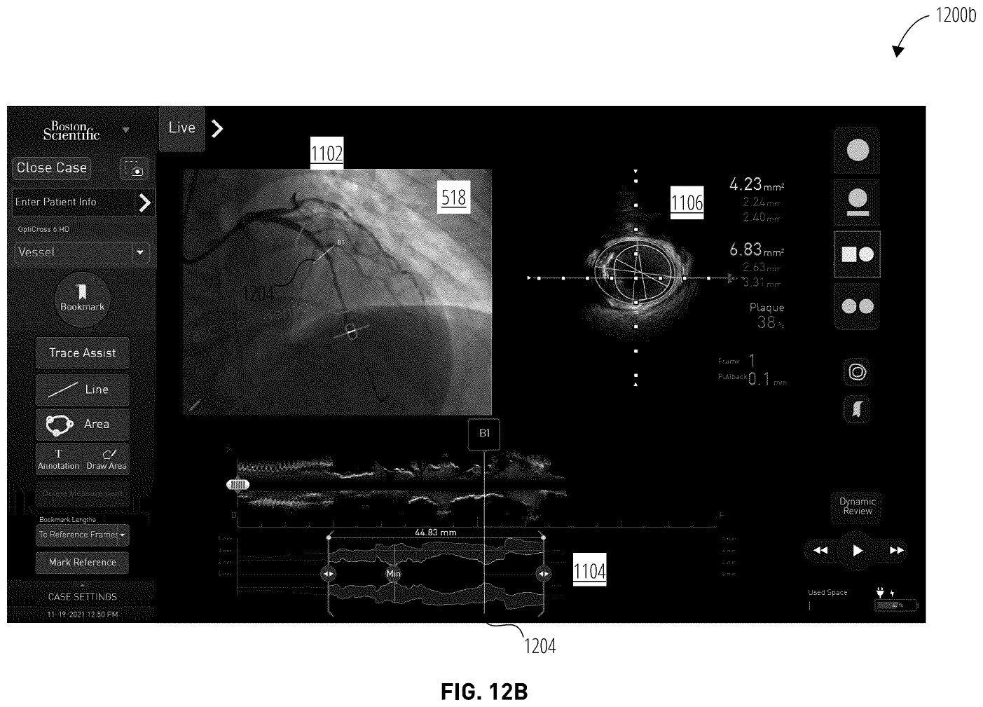

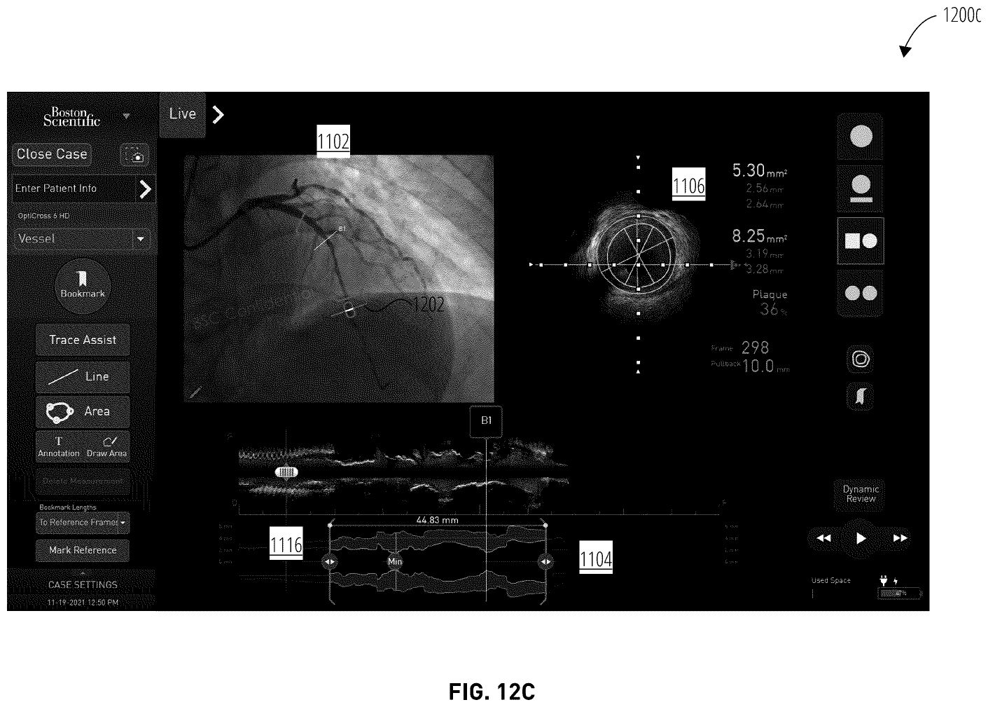

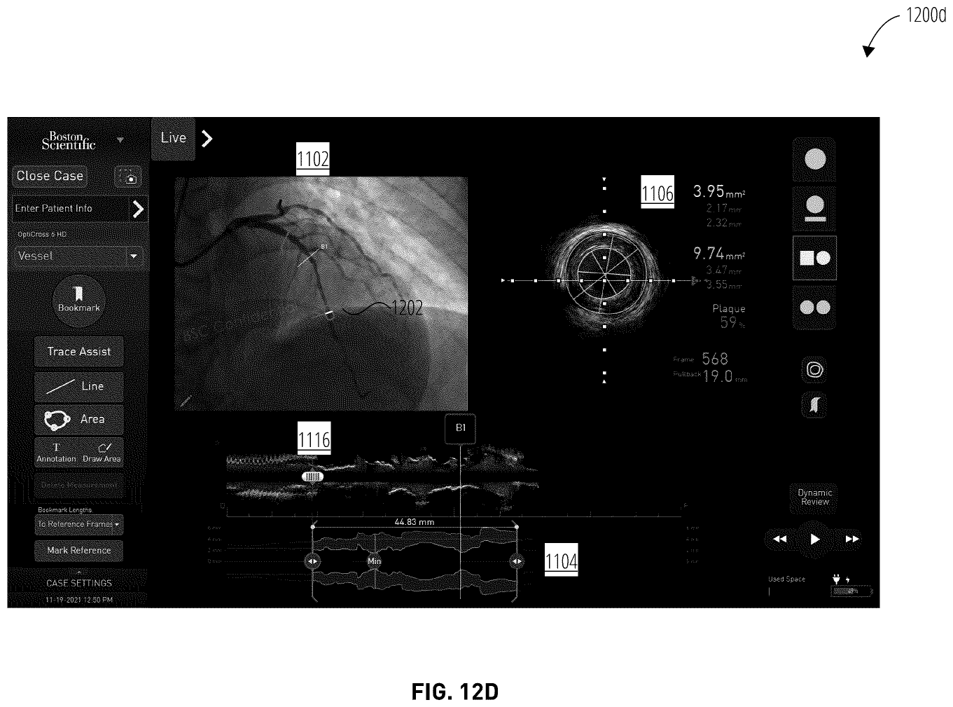

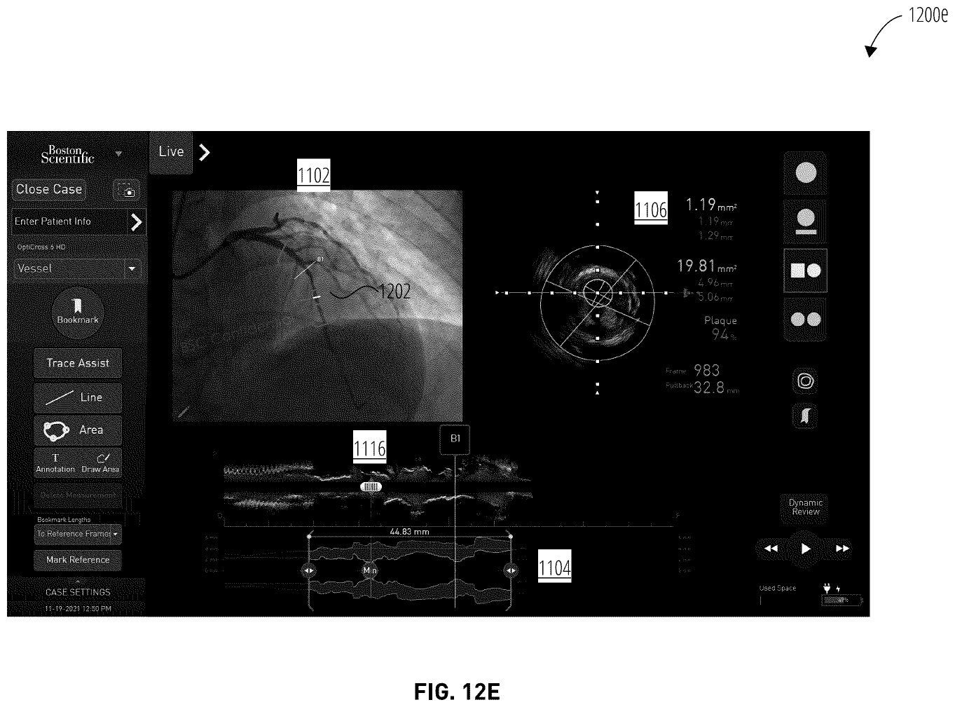

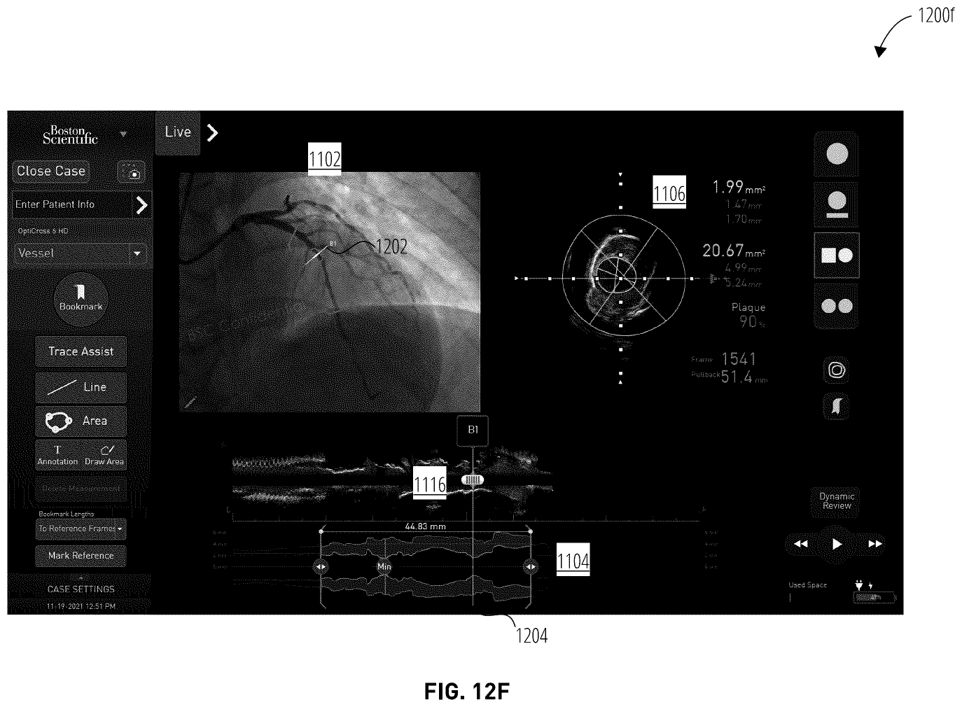

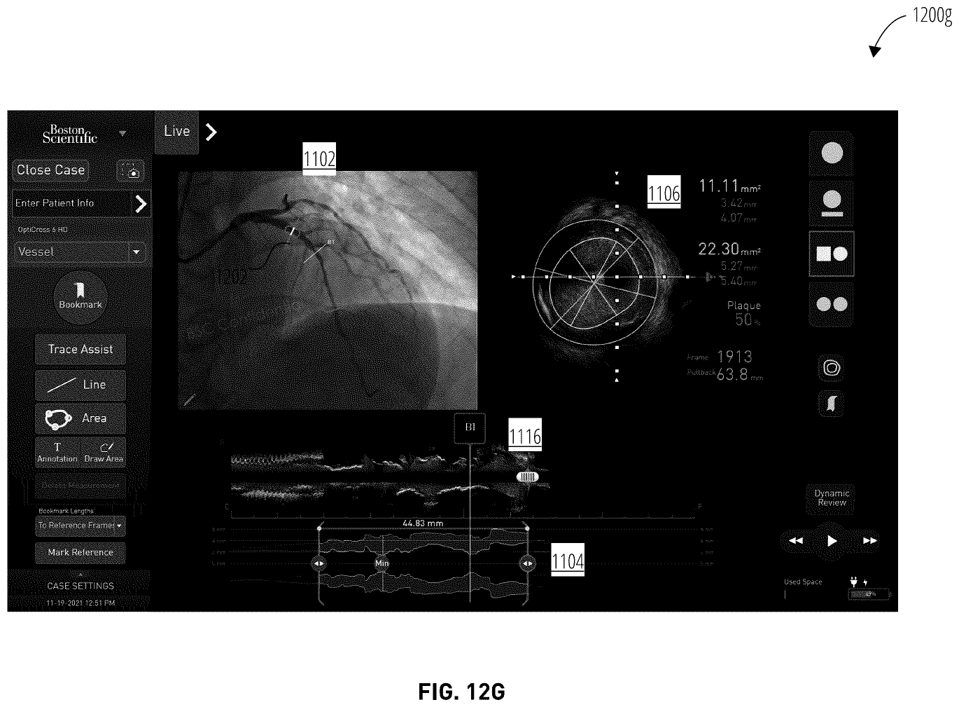

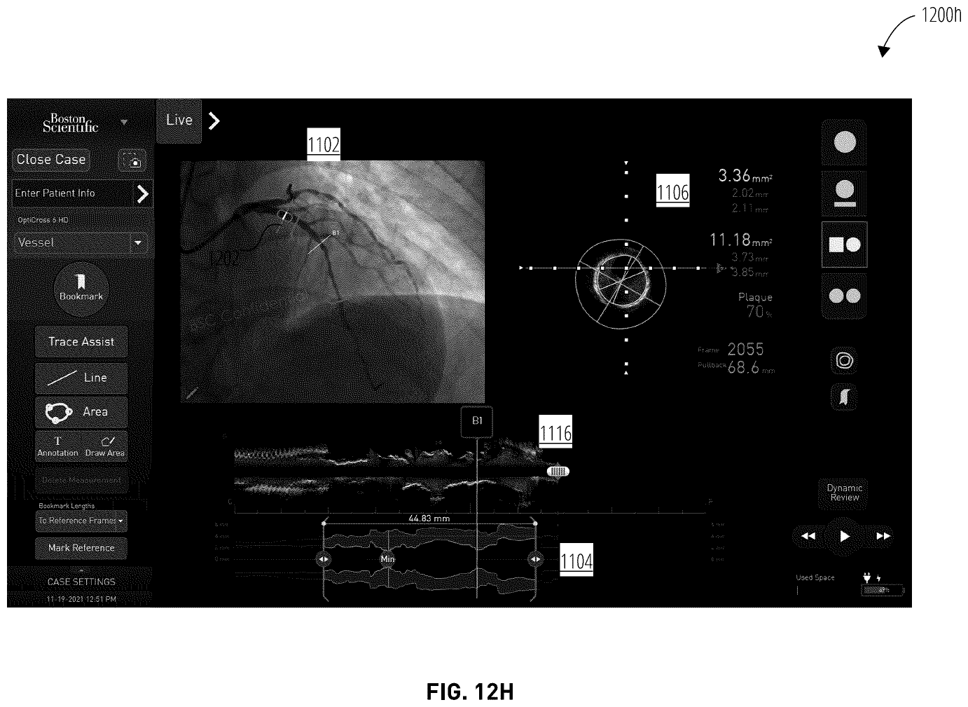

FIG. 12A through FIG. 12H illustrate GUI 1200a through GUI 1200h, respectively, each of which can be generated as GUI 530. Processor 504 can execute instructions 514 to generate GUI 530 comprising graphical components 520 like depicted in these GUIs. In general, these GUIs include graphical representations like GUI 1100a but with the results of the confirmed co-registration depicted in external image view 1102. These GUIs depict graphical representations of key frames 1134 disposed over external images 518 and a slider marker 1202 coupled with slider 1116. As such, processor 504 can execute instructions 514 to receive an indication to move slider 1116 (e.g., via I/O devices 508, or the like). Additionally, in some embodiments, a bookmark (or bookmarks) to frames of IVUS images 516 can be displayed in external image view 1102 and vessel profile view 1104. For example, FIG. 12B depicts GUI 1200b illustrating a graphical representation of bookmark 1204 displayed in external image view 1102 and vessel profile view 1104.

Responsive to receiving the indication, processor 504 can execute instructions 514 to generate the GUIs in these figures. As slider 1116 is moved in vessel profile view 1104, slider marker 1202 is moved in external image view 1102 and the frame of IVUS images 516 depicted in cross section view 1106 is adjusted accordingly. The benefit of co-registration being that the frames of IVUS images 516 are aligned with the external images 518 such that when slider 1116 is adjusted and slider marker 1202 indicates the location of the slider 1116 on external images 518, the frame of IVUS images 516 matching that location of the vessel 202 in external images 518 is displayed in cross section view 1106.

FIG. 12C illustrates GUI 1200c depicting the slider 1116 moved proximally along the vessel 202 and the location of slider marker 1202 in external image view 1102 and the frame of IVUS images 516 represented in cross section view 1106 adjusted accordingly. Processor 504 can execute instructions 514 to generate GUI 1200c as GUI 530 responsive to received input.

FIG. 12D illustrates GUI 1200d depicting the slider 1116 further moved proximally along the vessel 202 and the location of slider marker 1202 in external image view 1102 and the frame of IVUS images 516 represented in cross section view 1106 adjusted accordingly. Processor 504 can execute instructions 514 to generate GUI 1200d as GUI 530 responsive to received input.

FIG. 12E illustrates GUI 1200e depicting the slider 1116 further moved proximally along the vessel 202 and the location of slider marker 1202 in external image view 1102 and the frame of IVUS images 516 represented in cross section view 1106 adjusted accordingly. Processor 504 can execute instructions 514 to generate GUI 1200e as GUI 530 responsive to received input.

FIG. 12F illustrates GUI 1200f depicting the slider 1116 further moved proximally along the vessel 202 and the location of slider marker 1202 in external image view 1102 and the frame of IVUS images 516 represented in cross section view 1106 adjusted accordingly. Processor 504 can execute instructions 514 to generate GUI 1200f as GUI 530 responsive to received input.

FIG. 12G illustrates GUI 1200g depicting the slider 1116 further moved proximally along the vessel 202 and the location of slider marker 1202 in external image view 1102 and the frame of IVUS images 516 represented in cross section view 1106 adjusted accordingly. Processor 504 can execute instructions 514 to generate GUI 1200g as GUI 530 responsive to received input.

FIG. 12H illustrates GUI 1200h depicting the slider 1116 further moved proximally along the vessel 202 and the location of slider marker 1202 in external image view 1102 and the frame of IVUS images 516 represented in cross section view 1106 adjusted accordingly. Processor 504 can execute instructions 514 to generate GUI 1200h as GUI 530 responsive to received input.

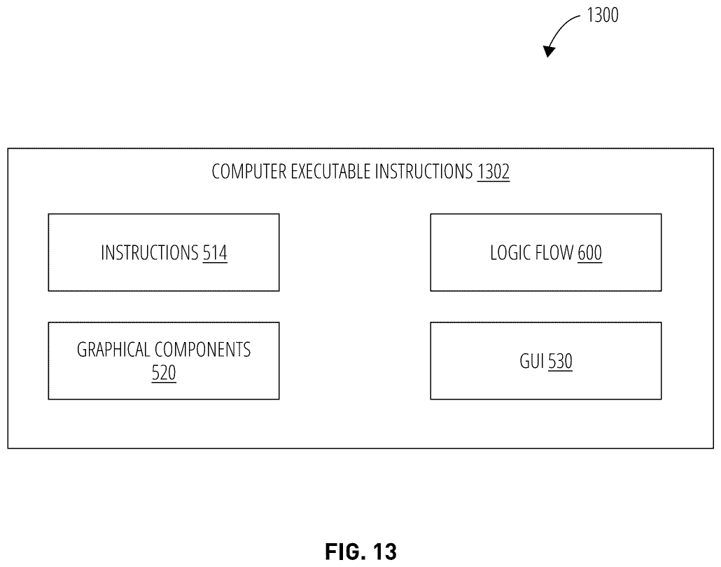

FIG. 13 illustrates computer-readable storage medium 1300. Computer-readable storage medium 1300 may comprise any non-transitory computer-readable storage medium or machine-readable storage medium, such as an optical, magnetic or semiconductor storage medium. In various embodiments, computer-readable storage medium 1300 may comprise an article of manufacture. In some embodiments, computer-readable storage medium 1300 may store computer executable instructions 1302 with which circuitry (e.g., processor 106, processor 504, IVUS/Angio imaging system acquisition circuitry 512, and the like) can execute. For example, computer executable instructions 1302 can include instructions to implement operations described with respect to instructions 514, logic flow 600, graphical components 520, and/or GUI 530. Examples of computer-readable storage medium 1300 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1302 may include any suitable type of code, such as source code, compiled code, interpreted code, executable code, static code, dynamic code, object-oriented code, visual code, and the like.

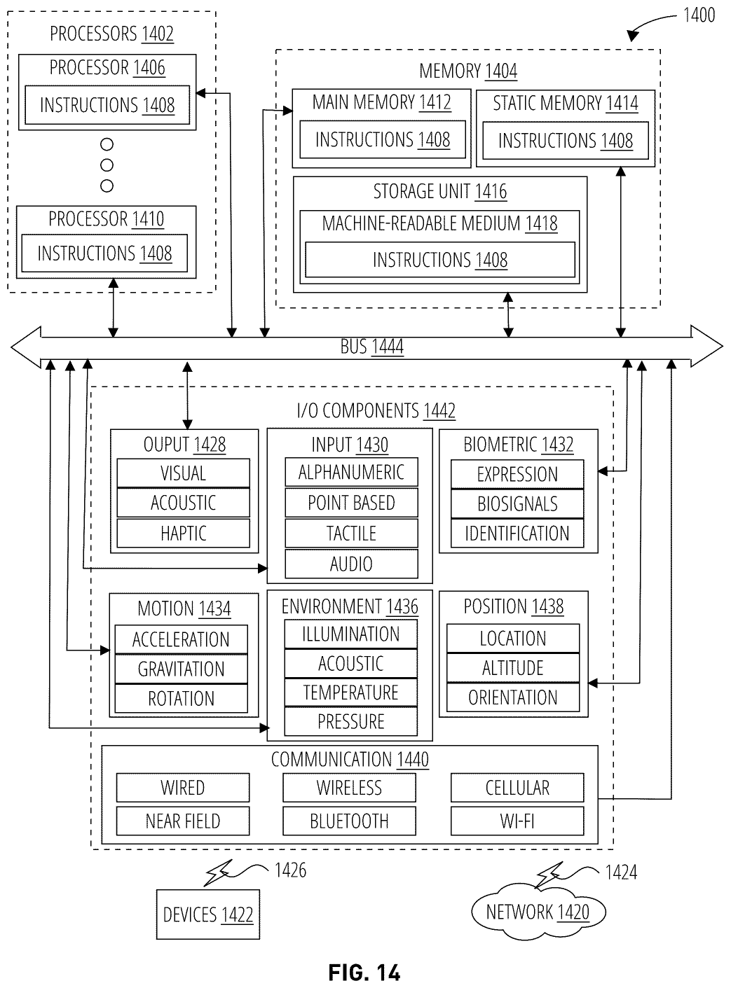

FIG. 14 illustrates a diagrammatic representation of a machine 1400 in the form of a computer system within which a set of instructions may be executed for causing the machine to perform any one or more of the methodologies discussed herein. More specifically, FIG. 14 shows a diagrammatic representation of the machine 1400 in the example form of a computer system, within which instructions 1408 (e.g., software, a program, an application, an applet, an app, or other executable code) for causing the machine 1400 to perform any one or more of the methodologies discussed herein may be executed. For example, the instructions 1408 may cause the machine 1400 to execute logic flow 600 of FIG. 6, or the like. More generally, the instructions 1408 may cause the machine 1400 to automatically determine key frames during a pre-PCI, peri-PCI, or post-PCI using IVUS. It is noted that the present disclosure provides specific and discrete implementations of co-registering IVUS images 516 and external images 518 even after acquisition, which is a significant improvement over the prior art.

The instructions 1408 transform the general, non-programmed machine 1400 into a particular machine 1400 programmed to carry out the described and illustrated functions in a specific manner. In alternative embodiments, the machine 1400 operates as a standalone device or may be coupled (e.g., networked) to other machines. In a networked deployment, the machine 1400 may operate in the capacity of a server machine or a client machine in a server-client network environment, or as a peer machine in a peer-to-peer (or distributed) network environment. The machine 1400 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1408, sequentially or otherwise, that specify actions to be taken by the machine 1400. Further, while a single machine 1400 is illustrated, the term “machine” shall also be taken to include a collection of machines 1400 that individually or jointly execute the instructions 1408 to perform any one or more of the methodologies discussed herein.

The machine 1400 may include processors 1402, memory 1404, and I/O components 1442, which may be configured to communicate with each other such as via a bus 1444. In an example embodiment, the processors 1402 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1406 and a processor 1410 that may execute the instructions 1408. The term “processor” is intended to include multi-core processors that may comprise two or more independent processors (sometimes referred to as “cores”) that may execute instructions contemporaneously. Although FIG. 14 shows multiple processors 1402, the machine 1400 may include a single processor with a single core, a single processor with multiple cores (e.g., a multi-core processor), multiple processors with a single core, multiple processors with multiples cores, or any combination thereof.

The memory 1404 may include a main memory 1412, a static memory 1414, and a storage unit 1416, both accessible to the processors 1402 such as via the bus 1444. The main memory 1404, the static memory 1414, and storage unit 1416 store the instructions 1408 embodying any one or more of the methodologies or functions described herein. The instructions 1408 may also reside, completely or partially, within the main memory 1412, within the static memory 1414, within machine-readable medium 1418 within the storage unit 1416, within at least one of the processors 1402 (e.g., within the processor's cache memory), or any suitable combination thereof, during execution thereof by the machine 1400.

The I/O components 1442 may include a wide variety of components to receive input, provide output, produce output, transmit information, exchange information, capture measurements, and so on. The specific I/O components 1442 that are included in a particular machine will depend on the type of machine. For example, portable machines such as mobile phones will likely include a touch input device or other such input mechanisms, while a headless server machine will likely not include such a touch input device. It will be appreciated that the I/O components 1442 may include many other components that are not shown in FIG. 14. The I/O components 1442 are grouped according to functionality merely for simplifying the following discussion and the grouping is in no way limiting. In various example embodiments, the I/O components 1442 may include output components 1428 and input components 1430. The output components 1428 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1430 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1442 may include biometric components 1432, motion components 1434, environmental components 1436, or position components 1438, among a wide array of other components. For example, the biometric components 1432 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1434 may include acceleration sensor components (e.g., accelerometer), gravitation sensor components, rotation sensor components (e.g., gyroscope), and so forth. The environmental components 1436 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1438 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1442 may include communication components 1440 operable to couple the machine 1400 to a network 1420 or devices 1422 via a coupling 1424 and a coupling 1426, respectively. For example, the communication components 1440 may include a network interface component or another suitable device to interface with the network 1420. In further examples, the communication components 1440 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1422 may be another machine or any of a wide variety of peripheral devices (e.g., a peripheral device coupled via a USB).

Moreover, the communication components 1440 may detect identifiers or include components operable to detect identifiers. For example, the communication components 1440 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1440, such as location via Internet Protocol (IP) geolocation, location via Wi-Fi® signal triangulation, location via detecting an NFC beacon signal that may indicate a particular location, and so forth.

The various memories (i.e., memory 1404, main memory 1412, static memory 1414, and/or memory of the processors 1402) and/or storage unit 1416 may store one or more sets of instructions and data structures (e.g., software) embodying or utilized by any one or more of the methodologies or functions described herein. These instructions (e.g., the instructions 1408), when executed by processors 1402, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1420 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1420 or a portion of the network 1420 may include a wireless or cellular network, and the coupling 1424 may be a Code Division Multiple Access (CDMA) connection, a Global System for Mobile communications (GSM) connection, or another type of cellular or wireless coupling. In this example, the coupling 1424 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1408 may be transmitted or received over the network 1420 using a transmission medium via a network interface device (e.g., a network interface component included in the communication components 1440) and utilizing any one of several well-known transfer protocols (e.g., hypertext transfer protocol (HTTP)). Similarly, the instructions 1408 may be transmitted or received using a transmission medium via the coupling 1426 (e.g., a peer-to-peer coupling) to the devices 1422. The terms “transmission medium” and “signal medium” mean the same thing and may be used interchangeably in this disclosure. The terms “transmission medium” and “signal medium” shall be taken to include any intangible medium that can store, encoding, or carrying the instructions 1408 for execution by the machine 1400, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

By using genuine models of anatomy more accurate surgical plans may be developed than through statistical modeling.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An apparatus for an intravascular ultrasound (IVUS) imaging system, comprising:
a display;
an interface configured to couple to an IVUS catheter;
a processor coupled to the interface and the display; and
a memory device comprising instructions, which when executed by the processor cause the IVUS imaging system to:
receive an indication of an end location, the end location corresponding to a location of an intravascular ultrasound (IVUS) guide catheter distal tip on an external image, wherein the IVUS guide catheter is or was inserted into a vessel and the vessel is represented in the external image;
register, based in part on the location of the IVUS guide catheter distal tip and a start location, each of a plurality of frames of a series of IVUS images captured from within the vessel via an IVUS pullback operation to locations on the external image to generate a pullback path of the IVUS pullback operation and a branch, wherein the start location corresponding to a location of the start of the IVUS pullback operation;
generate a graphical user interface (GUI) comprising indications of the external image, the start location, the end location, the pullback path, and the branch;
receive, via an input device and the GUI at least one modification to the pullback path and/or the branch;
regenerate the GUI, the regenerated GUI comprising indications of:
the external image, the start location, the end location, and
the pullback path and the modified branch,
the modified pullback path and the branch, or
the modified pullback path and the modified branch; and
receive, via the input device and the regenerated GUI a confirmation that the modified pullback path and/or the modified branch is confirmed.

2. The apparatus of claim 1, wherein the at least one modification is a modification to the pullback path and wherein the memory device further comprises instructions that when executed by the processor cause the IVUS imaging system to:
receive, via the input device and the regenerated GUI a modification to the branch; and
regenerate the GUI a second time, the second regenerated GUI comprising indications of the external image, the start location, the end location, the modified pullback path, and the modified branch.

3. The apparatus of claim 2, wherein the pullback path is registered to a longitudinal axis of the vessel.

4. The apparatus of claim 3, wherein the pullback path comprises a plurality of nodes and wherein the at least one modification to the pullback path comprises a modification to a location of one or more of the plurality of nodes.

5. The apparatus of claim 4, wherein the modification to the branch changes a location of the branch along the longitudinal axis.

6. The apparatus of claim 5, wherein the branch is a first branch of a plurality of branches and wherein the memory device further comprises instructions that when executed by the processor cause the IVUS imaging system to change the location of a second branch of the plurality of branches responsive to the modification.

7. The apparatus of claim 6, wherein the first regenerated GUI further comprises numerical or textual designations for the plurality of branches.

8. The apparatus of claim 1, the memory device further comprises instructions that when executed by the processor cause the IVUS imaging system to:
retrieve an angiogram from a memory device, the angiogram comprising a plurality of frames;
generate an external image selection GUI comprising an indication of a frame of the plurality of frames of the angiogram and at least one navigation button to scroll through plurality of frames; and
receive, via the input device and the external image selection GUI, an indication to select a one of the plurality of frames of the angiogram as the external image.

9. The apparatus of claim 1, the memory device further comprises instructions that when executed by the processor cause the IVUS imaging system to receive, from the input device, an indication of the start location.

10. The apparatus of claim 9, the memory device further comprises instructions that when executed by the processor cause the IVUS imaging system to:
receive, from the input device, an indication of the start location on an external image of the vessel;
capture the external image via external image acquisition circuitry; and
capture the series of IVUS images via IVUS image acquisition circuitry.

11. At least one machine readable storage device, comprising a plurality of instructions that in response to being executed by a processor of an intravascular ultrasound (IVUS) imaging system cause the processor to:
receive an indication of an end location, the end location corresponding to a location of an intravascular ultrasound (IVUS) guide catheter distal tip on an external image, wherein the IVUS guide catheter is or was inserted into a vessel and the vessel is represented in the external image;
register, based in part on the location of the IVUS guide catheter distal tip and a start location, each of a plurality of frames of a series of IVUS images captured from within the vessel via an IVUS pullback operation to locations on the external image to generate a pullback path of the IVUS pullback operation and a branch, wherein the start location corresponding to a location of the start of the IVUS pullback operation;
generate a graphical user interface (GUI) comprising indications of the external image, the start location, the end location, the pullback path, and the branch;
receive, via an input device and the GUI at least one modification to the pullback path and/or the branch;
regenerate the GUI, the regenerated GUI comprising indications of:
the external image, the start location, the end location, and
the pullback path and the modified branch,
the modified pullback path and the branch, or
the modified pullback path and the modified branch; and
receive, via the input device and the regenerated GUI a confirmation that the modified pullback path and/or the modified branch is confirmed.

12. The at least one machine readable storage device of claim 11, further comprising instructions that in response to being executed by the processor cause the processor to:
receive, via the input device and the regenerated GUI a modification to the branch; and
regenerate the GUI a second time, the second regenerated GUI comprising indications of the external image, the start location, the end location, the modified pullback path, and the modified branch.

13. The at least one machine readable storage device of claim 11, further comprising instructions that in response to being executed by the processor cause the processor to:
receive, from the input device, an indication of the start location on an external image of the vessel;
capture the external image via external image acquisition circuitry; and
capture the series of IVUS images via IVUS image acquisition circuitry.

14. The at least one machine readable storage device of claim 11, wherein the external image is an x-ray image.

15. The at least one machine readable storage device of claim 11, wherein the regenerated GUI further comprises an indication of a longitudinal view of the series of IVUS images, wherein the first regenerated GUI further comprises a slider to scroll along the longitudinal axis of the series of IVUS images and a slider marker dispo sed on the pullback path, wherein the slider marker is linked to the slider such that movement of the slider causes a corresponding movement of the slider marker on the pullback path.

16. A method, comprising:
receiving an indication of an end location, the end location corresponding to a location of an intravascular ultrasound (IVUS) guide catheter distal tip on an external image, wherein the IVUS guide catheter is or was inserted into a vessel and the vessel is represented in the external image;
registering, based in part on the location of the IVUS guide catheter distal tip and a start location, each of a plurality of frames of a series of IVUS images captured from within the vessel via an IVUS pullback operation to locations on the external image to generate a pullback path of the IVUS pullback operation and a branch, wherein the start location corresponding to a location of the start of the IVUS pullback operation;
generating a graphical user interface (GUI) comprising indications of the external image, the start location, the end location, the pullback path, and the branch;
receiving, via an input device and the GUI at least one modification to the pullback path and/or the branch;
regenerating the GUI, the regenerated GUI comprising indications of:
the external image, the start location, the end location, and
the pullback path and the modified branch,
the modified pullback path and the branch, or
the modified pullback path and the modified branch; and
receiving, via the input device and the regenerated GUI a confirmation that the modified pullback path and/or the modified branch is confirmed.

17. The method of claim 16, wherein the at least one modification is a modification to the pullback path, the method comprising:
receiving, via the input device and the regenerated GUI a modification to the branch; and
regenerating the GUI a second time, the second regenerated GUI comprising indications of the external image, the start location, the end location, the modified pullback path, and the modified branch.

18. The method of claim 17, wherein the pullback path is registered to a longitudinal axis of the vessel.

19. The method of claim 18, wherein the pullback path comprises a plurality of nodes and wherein the at least one modification to the pullback path comprises a modification to a location of one or more of the plurality of nodes.

20. The method of claim 19, wherein the modification to the branch changes a location of the branch along the longitudinal axis.
